(19) United States
(12) Patent Application Publication
(10) Pub. No.: US 2006/0025458 A1
(43) Pub. Date: Feb. 2, 2006

(54) Alkylammonium compounds as antifungal and antitrypanosomal agents

(75) Inventor: Choukri B. Mamoun, Farmington, CT (US)

Correspondence Address: EDWARDS & ANGELL, LLP, P.O. BOX 55874, BOSTON, MA, 02205, US

(73) Assignee: University of Connecticut

(21) Appl. No.: 11/186,658

(22) Filed: Jul. 21, 2005

Related U.S. Application Data
(60) Provisional application No. 60/592,551, filed on Jul. 30, 2004.

(51) Int. Cl. A61K 31/426; A61K 31/40; A61K 31/14

(52) U.S. Cl. 514/365; 514/408; 514/643

(57) ABSTRACT

The use of alkyl quaternary ammonium compounds including certain choline analogs for treating or preventing fungal and trypanosomal (e.g., Leishmaniasis) infections is described. These compounds, characterized as mono- and bis-alkyl ammonium compounds, were demonstrated to be highly effective in inhibiting growth of Candida albicans, Saccharomyces cerevisiae and Leishmania major. Quaternary ammonium compounds were previously known as effective antimalarial compounds in vivo but not recognized as antifungals or as anti-trypanosomals (e.g., anti-Leishmanials).

### REFERENCE TO RELATED APPLICATIONS

This application claims benefit to Provisional Application Ser. No. 60/592,551 filed Jul. 30, 2004.

### BACKGROUND OF THE INVENTION

Fungal infections are caused by organisms called fungi that exist as single cells but under special conditions can also undergo a morphological change to form chains of cells. Common fungal infections include athlete's foot, jock itch, ringworm and candidiasis, also called thrush or yeast infection). Candidiasis is caused by species of the genus Candida. One of these species, Candida albicans, causes recalcitrant infections of skin, oral, gastrointestinal and urogenital systems, and is the leading cause of invasive fungal disease in premature infants, diabetics, surgical patients, trauma patients and immunocompromised hosts. Mortality from this species ranges from 30 to 50% in immunocompromised patients (Viudes, et al., 2002). Fungal infections are commonly found in the mouth, armpits, groin and genital areas, but can also be found in other parts of the body. The symptoms include itching, burning and cracked skin.

For treatment of skin infections, various topical antifungal drugs are available and exist in various forms, including creams, ointments, liquids, powders, aerosol sprays, and vaginal suppositories. Among the topical antifungal are ciclopirox, clotrimazole, econazole, miconazole, nystatin, oxiconazole, terconazole, and tolnaftate. Among the brands of products that contain topical antifungal drugs are Absorbine Jr., Desenex, Gyne-Lotrimin, Loprox, Lotrimin, Micatin, Monistat, Mycelex, Mycolog-II, Oxistat, Spectazole Cream, Terazol, and Tinactin. Products with the same brand name may not contain the same active ingredient. Certain topical antifungal drugs may be more effective than others against particular types of fungal infections. For example, some may work well for treating athlete's foot, but not for treating a yeast infection.

Fungi are characterized as eukaryotes having a rigid cell wall composed of chitin and polysaccharides. They are resistant to most antibacterial agents and the anti-fungal drugs currently in use tend to be quite toxic, thus limiting use for systemic infections.

Subcutaneous and Systemic Mycotic Infections

The following compounds are among the more popular antifungal compounds currently in use.

Amphotericin B (Fungizone®)

Amphotericin B is a polyene antibiotic having multiple double bonds and is used to treat systemic mycoses, despite its toxic potential. It is sometimes given in combination with flucytosine to limit toxicity so that lower doses can be administered.

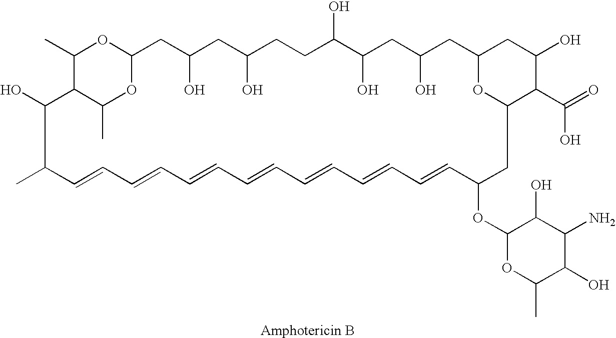

Amphotericin B binds to ergosterol in the fungal plasma membrane, and forms channels. This disrupts membrane function, allowing electrolytes, especially K+, to leak out of the cell, resulting in cell death. The polyene antibiotics in general bind preferentially to ergosterol, which is the main steroid in fungal membranes.

Amphotericin B is either fungicidal or fungistatic, depending on the pathogen and drug concentration. It is effective against Candida albicans, histoplasma capsulatum, cryptococcus neoformans, coccidioides immitus, aspergillus nidulans and blastomyces dermatitidis.

The drug is administered by IV infusion. It is highly bound to tissues and plasma proteins and can displace other drugs. Other characteristics include a long half-life (about 2-weeks), inaccessibility to CNS, even at sites of inflammation. For access to CNS, it must be given by intrathecal route. It is neither metabolized by liver, nor excreted by kidneys. Most excretion is biliary.

Adverse effects include a low therapeutic index (high potential for toxicity), fever and chills during IV administration, and renal impairment occuring in 80% of patients. The drug accumulates in kidneys, disrupting cell membranes and in high doses can cause irreversible damage. Hypertension may be serious and occur as a shock-like drop in blood pressure. Drug use has also been associated with hypokalemia, where elevated extracellular K+ is excreted, necessitating K+ supplementation. Normochromic, normocytic anemia caused by a reversible suppression of erythrocyte production may also occur.

Flucytosine (Ancobnon®) Capsules

Flucytosine is a pyrimidine analog. It is used only in combination with amphotericin B for systemic infections, with the exception it may be used alone for subcutaneous chromomycosis.

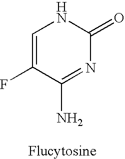

The drug enters fungal cells via a cytosine-specific permease, which is part of a pore-forming membrane transport protein. It disrupts DNA synthesis. In the fungal cell, flucytosine is converted to 5-fluorodeoxyuridylic acid (5-FdUMP) which inhibits thymidylate synthetase, thereby depriving the cell of thymidine necessary for DNA synthesis. It also disrupts protein synthesis where it is also converted to 5-fluorouridine triphosphate (5-FUTP) which is incorporated into fungal RNA, and disrupts protein synthesis.

The antifungal spectrum includes Candida, Cryptococcus, Aspergillus, among others. Resistance can develop during long-term therapy, so fluocytosine is seldom used alone, most often used in combination with amphotericin B.

Adverse effects include bone marrow suppression (hematological toxicity, due to metabolite, 5-fluorouracil), hepatic dysfunction (partial hepatic metabolism to 5-fluorouracil) and GI distress.

Ketoconazole (Nizoral″) Tablets

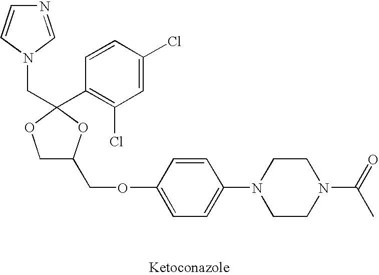

Ketoconazole is useful for treating systemic and subcutaneous infections. It also inhibits gonadal and adrenal steroid synthesis, resulting in suppression of testosterone and cortisol synthesis.

Ketoconazole blocks ergosterol synthesis by inhibiting the P450 catalyzed conversion of lanosterol to ergosterol (the main steroid in fungal membranes). Inhibition of ergosterol synthesis disrupts membrane function and increases permeability.

The compound is either fungistatic or fungicidal, depending on pathogen and dose. It is currently the most effective treatment for histoplasmosis. It is also effective against non-meningitis, Cryptococcus, Blastomyces, Candida, and various dermatophytic infections; e.g., Tinea infections.

The drug is orally administered and requires an acidic stomach pH for absorption. It should not be taken with antacids, H₂ blockers, or omperazole (proton-pump inhibitor). It does not enter CNS and is therefore not effective for fungal meningitis. It causes cytochrome P-450 induction and is excreted mostly in bile.

Adverse effects of ketoconazole include gynecomastia (in men), caused by blocking of androgen synthesis.

Ketoconazole should not be administered with Amphotercin B. Amphotericin B needs ergosterol to be active and Ketoconazole inhibits ergosterol synthesis. Contraindications also apply to administration with other drugs, which require P-450 metabolism and drugs which reduce stomach pH.

Ketoconazole is synergistic with fluocytosine when used against Candida infections.

Fluconazole (Diflucan® Tablets or IP)

Fluconazole is a more recent drug in the imidazole series (fluorine substituted, not chlorine like others). Like ketoconazole, it inhibits ergosterol synthesis; however, it differs from ketoconazole in that it can penetrate CNS (effective against fungal meningitis), does not require an acid pH in the stomach for absorption, shows significantly less P-450 induction and most elimination is renal, unmetabolized.

The spectrum is different from ketoconazole, making it the drug of choice for cryptococcal meningoencephilitis, histoplasmosis, and coccidomycosis (in immunocompromized, AIDS patients). It also inhibits Histoplasma, Cryptococcus, Blastomyces, and Candida; however, it is not effective against Aspergillis or other filamentous fungi.

Superficial Mycoses

Drugs used in the treatment of superficial mycoses include Griseofulvin, Nystatin, Miconazole, Clotrimazole, Clotrimazole, Econazole and Tolnaftate.

Griseofulvin (oral administration) was isolated from Penicillium griseofulvum in 1939.

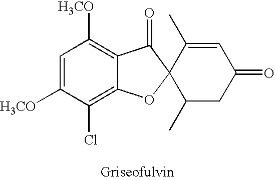

Griseofulvin exhibits a colchicine-like action, but is fungal-specific. It enters fungal cells by active transport and interferes with microtubule assembly and inhibits mitosis by interfering with mitotic spindle formation. Griseofulvin concentrates in keratinized tissues; e.g., skin, hair, nails, making them unsuitable for fungal growth. Therapy must be provided until normal tissue replaces infected tissue, typically requiring months of therapy.

The drug is orally administered for cutaneous infections and is not effective topically. It distributes to keratinized tissues, inclduding skin, hair and nails.

Metabolites of the drug are renally eliminated. Cytochrome P450 is induced in the liver.

While fairly safe, adverse effects include allergic reactions, headache, nausea and potentiation of ethanol intoxication. It is contraindicated in patients with intermittent porphyria, a condition presenting with elevated heme synthesis and high Fe-protoporphyrin levels in the blood. Drugs that induce cytochrome P-450, which is a heme-protein, also tend to induce heme biosynthesis, leading to elevated amounts of circulating heme. A P-450 inducing drug should not be administered to patients with intermittent porphyria.

Nystatin is a polyene antibiotic with a structure and mechanism similar to amphotericin B. It binds to ergosterol in fungal membranes, disrupts membrane functions and increases permeability. It is used topically for treatment of cutaneous and mucosal Candida infections, but is not used for systemic infections because of high toxicity. It is never administered parenterally. Because it is not absorbed orally, it may be given orally to treat local oral thrush and intestinal candidiasis.

Miconazole, clotrimazole, and econazole are to pical drugs that are rarely administered parenterally because of their high toxicity. They are used for oral, vaginal, or cutaneous Candida infections, in the form of creams or troches.

Tolnaftate (Aftate®) (Tinactin®) is a topical drug that is effective against dermatophytes such as Tinea and Microsporum. It is not effective against Candida. The mechanism of action is not known; however, there is no known toxicity.

Fungal infections can be topical or systemic. The following are examples of the many species for which different drugs are used with varying degrees of success: Candida, Aspergillus, Coccididomycosis (Coccidioides immitis), Filobasidiella neoformans, Blastomycesdermatitidis, Paracoccidioides bresiliensis, Sporothrix schenckii, hormodendrum pedrosoi and Rhinosporidium seeberi.

Candida albicans, for example, is an opportunistic and dimorphic pathogenic fungus that is able to cause recalcitrant infections of skin, oral, gastrointestinal and urogenital systems. Depending on host immunity, infection by this organism can be superficial or can be hematogenously disseminated, resulting in life-threatening systemic candidiasis. The limited arsenal of antifungal agents, high toxicity exhibited by some of those drugs, and emergence of resistance, emphasize the need for new antifungal compounds.

Parasitic Infections

Leishmaniasis is one of many protozoan parasite diseases that is particularly common in undeveloped countries but is found also in other parts of the world. While it is often manifest on the skin, giving rise to ulcerated skin lesions, the condition may also be visceral. Closely related is Trypanosoma, both of which cause a number of human diseases, including Chagas Disease, Leishmaniasis and African Sleeping Sickness. Current treatments for these diseases are generally ineffective, impractical or highly toxic.

Deficiencies in the Art

Deficiencies in currently used therapies to treat fungal and Leishmaniasis infections signify a need for new antifungal drugs that are effective against a wide range of these organisms, and importantly, are low in toxic side effects. Effective antifungal and anti-leishmaniasis drug should not only relieve the symptoms but also clear the site of infection. Unfortunately, many compounds used to treat these conditions cannot be used to treat systemic infections due to toxicity.

Fungal infections can be topical or systemic. The following are examples of the many species for which different drugs are used with varying degrees of success: Candida, Aspergillus, Coccididomycosis (Coccidiodes immitis), Filobasidiella neoformans, Blastomycesdermatitidis, Paracoccidioides bresiliensis, Sporothrix schenckii, hormodendrum pedrosoi and Rhinosporidium seeberi.

Candida albicans, for example is an opportunistic and dimorphic pathogenic fungus that is able to cause recalicitrant infections of skin, oral, gastrointestinal and urogenital systems. Depending on host immunity, infection by this organism can be superficial or hematogenously disseminated, resulting in life-threatening systemic candidiasis. The limited arsenal of antifungal agents, high toxicity exhibited by some of these drugs, and emergence of resistance emphasize the need for new antifungal compounds.

### SUMMARY OF THE INVENTION

The invention is related to the unexpected discovery that several alkyl mono-and bis-ammonium compounds previously associated with anti-malarial activity, exhibit high activity against fungi and against the parasitic protozoan Leishmania major. In particular, significant in vitro activity has been shown against Candida albicans and Saccharomyces cerevisiae

The results with these different fungi indicate that compounds previously used as antimalarials will be useful in treating mammalian fungal and leishmanial infections and because of their low toxicity, will be suitable for both topical and systemic use. A study of the structure-activity relations in tests on model fungi indicates that the length of the alkyl bridge in the bis-analogs or length of an alkyl group on the nitrogen of the mono-quaternary ammonia analogs is an important structural factor, with long-chain alkyl groups (e.g., 12-18) appearing to be superior to short chain alkyls.

The discovery of antifungal and anti-Leishmanial activity of these compounds arose in part during studies on the mode of action of the antimalarial choline analog 1,16-hexadecamethylene bis-[N-methylpyrrolidinium] dibromide (DTAB) in P. falciparum and S. cerevisiae as part of an effort to determine the role and mechanism of action in choline transport. The initial objective was to use the yeast as a model for studying and identifying optimal antimalarials in treating multidrug resistant malaria and possibly other parasitic infections in addition to P. falciparum. An unexpected observation was the inhibition of yeast growth by DTAB. Further studies showed inhibition of growth of S. cerrvisiae, Candida albicans and Leishmania major by this compound with IC₅₀s ranging between 100 and 500 nM. Additional measurements with DTAB analogs showed antifungal and antileishmanial acitivity with inhibitions between 150 nM and 3 μM.

A number of alkyl ammonium compounds (kindly provided by Dr. Henri Vial, University of Montpellier II, France) were tested and found to exhibit antifungal activity. These compounds are identified herein as E2a, E6, E9, E13, E24, F4, G2, G4, G5, G14, G15, G25, H5, L1, L4, M34, M53, MS1, T3 and T4 and correspond to the compounds shown in FIG. 1.

As disclosed herein, activity against Leishmania was found with DTAB and with DTAB analogs. This protozoan parasite is the cause of cutaneous Leishmaniasis, which is most typically found in Asian countries, Africa and the Mediterranean basin. A visceral form of Leishmaniasis is prevalent in these areas as well as in several central and South American countries where untreated cases may have a fatality rate of 90%.

A large number of other reported antimalarial compounds are also expected to show the unexpected antifungal activity exhibited by the series of alkyl bis-ammonium compounds tested herein. Examples include molecules containing two quaternary ammonium groups on the ends of a hydrocarbon chain, which in particular have shown strong antimalarial and antibabesiosis activity such as set forth in U.S. Pat. No. 6,096,788, herein incorporated by reference in its entirety. In like manner, it is expected that the substituted bis-2-aminopyridines proposed for use in controlling parasitic infections will be useful as antifungals. A large number of exemplary compounds are set forth in U.S. Pat. No. 5,834,491, incorporated by reference in its entirety.

It is further contemplated that quaternary bis-ammonium salt precursors as well as certain related nitrogenous compounds; e.g., choline analogs, may have utility in vivo as prodrugs, analogous to the compounds disclosed in the International Publications WO 01/05742 and WO 2004/009068, each incorporated herein by reference in its entirety.

The invention in one aspect is a method for treating or preventing a fungal infection in a host, particularly in mammals and more particularly in humans. The therapy or prophylaxis is accomplished by administering an appropriately effective amount of an alkyl mono- or bis-quaternary ammonium compound in a pharmaceutically acceptable composition to a host in need thereof. The compound is selected from among one or more of the compounds represented by formula I:
R₁R₂R₃N⁺(CH₂)ₙR mX⁻ I
where R is hydrogen, phenyl, alkyl, alkenyl, alkynyl, alkylimine or R₁R₂R₃N⁺—, R₂ and R₃ can combine with the quaternary nitrogen to form a heterocyclic ring selected from the group consisting of pyrrolidine, pyrrole, pyrimidine, pyridine, thiazole, thiophene, thianyl, oxolanyl, imidazole and substituted derivatives thereof where the substituents are selected from the group consisting of alkyl and hydroxyalkyl C₁-C₅; n is 1-18, m is 1 or 2 and X⁻ is halide, tosylate or pharmaceutically acceptable esters, salts, solvates, clathrates or prodrugs thereof.

Particularly useful compounds include those compounds having the formula II:
R₁R₂R₃N⁺(CH₂)nN⁺R₄R₅R₆ mX⁻ II
where n is 2-18, R₁R₂R₃R₄R₅R₆ are independently alkyl, alkenyl or alkynyl; except when R₁—R₂ or R₄—R₅ are methylene; R₃ and R₆ are CH₂CH₂OH or CH₂CH₂OCH₃; m is 1 or 2 and X⁻ is halide, tosylate or a pharmaceutically acceptable salt thereof.

Other useful alkyl bis-quaternary ammonium compounds include 1,16-hexadecylmethylenebis-[N-methylpyrrolidine], 1,12-dodecanemethylene bis[4-methyl-5-ethylthiazoline] and their pharmaceutically acceptable salts. The alkyl bis-quaternary ammonium compound N,N,N,N- tetraethyl-N,N-di(2-hydroxyethyl)-1,16-hexadecanediaminium dibromide and 1,16-hexadecamethylene bis-[N-methylpyrrolidinium]dibromide (DTAB) are particularly preferred.

The invention also includes use of alkyl mono-quaternary ammonium compounds, such as those of formula III:
R₁R₂R₃N⁺(CH₂)n X⁻ III
where n is 2-18, R₁R₂R₃ are independently alkyl or alkenyl; R₁ is alkyl when R₁—R₂ is methylene; R₃ is —CH₂CH₂OH or CH₂CH₂OCH₃ when R₁ and R₃ are alkyl; and X⁻ is halide, tosylate or a pharmaceutically acceptable salt.

Particularly preferred mono-quaternary ammonium compounds include 1-dodecanemethylene[N-methylpyrrolidine] or trimethyl-octadecylmethylene-amidine and salts thereof.

The host is generally a mammal, most often a human. The disclosed compounds are useful when formulated as compositions for treating fungal skin infections in humans as well as in dogs and cats, or rodents such as rats and mice. Many other animals in zoos and as pets in home environments are susceptible to fungal infections and will benefit from treatment with compositions that include one or more of the described mono-or bis-quaternary ammonium compounds.

The disclosed method may be employed for treating infections caused by any of a number of fungi, including Candida, Aspergillus, Coccididomycosis (Coccidioides immitis), Filobasidiella neoformans, Blastomycesdermatitidis, Paracoccidioides bresiliensis, Sporothrix schenckii, hormodendrum pedrosoi and Rhinosporidium seeberi and is particularly suitable for Candida albicans or Saccharomyces cerevisiae infections.

The invention also includes antifungal compositions comprising one or more mono-or bis-quaternary alkyl ammonium compounds represented by formula IV:
R₁R₂R₃N⁺(CH₂)nN⁺R₁R₂R₃ mX⁻ IV
where R₁R₂R₃R₄R₅R₆ are independently alkyl, alkenyl or alkynyl; except when R₁—R₂ and R₄—R₅ are independently methylene, R₃ and R6 are independently alkyl; m is 1 or 2, n is 6-18, and X⁻ is halide, tosylate or a pharmaceutically acceptable salt.

Particularly preferred antifungal compositions may contain 1,12-dodecanemethylene bis[4-methyl-5-ethylthiazolium]diiodide and may include additional mono-or bis quaternary alkyl ammonium compounds such as any of those represented by formula I.

The compositions may also include additional compounds for therapeutic purposes, such as an anti-inflammatory, analgesic or anti-pyretic agent. Depending on medical indications, the antifungal may be administered in conjunction with agents such as those used in HIV treatments, hypertension or diabetes.

A particularly desirable property of the alkyl mono-or bis-quaternary ammonium choline analog compounds that may be used in practicing the methods of the invention is their lack of toxicity. In general these analogs consist of a long chain fatty acid having from 8-16 carbon atoms substituted on either one or the other end with a quaternary nitrogen. Such analogs can be administered systemically because of the low toxicity.

The compositions of the invention may be administered systemically or orally, depending on the formulation. Systemic administration may be intravenous, intramuscularly, interperitoneally, subcutaneously or by any well-recognized method of systemic administration.

Oral formulations are particularly preferred and may be comprised in suitable dosage form within a tablet. Timed release formulations are often desirable, as this often avoids a bolus effect and assures a more consistent therapeutic blood level. Liquid preparations are often preferable for children in order to ease stress in the administration process.

Suitable dosages can be readily determined by those skilled in the art and can be adjusted for adult and pediatric formulations.

In many instances, the preferred method of administration will be topical and the disclosed compositions may be conveniently applied as an ointment, cream or oil to an affected area. Many fungal infections occur on the skin and are readily treated with topical formulations.

The described pharmaceutical compositions are expected to be of particular benefit in as anti-trypanosomal or anti-Leishmanials. Such compositions may include one or more of the compounds of formula I.

Methods of treating fungal infections can be accomplished by administering to a host in need of therapy or prophylaxis thereof, an effective amount of a pharmaceutically acceptable composition that included any of the group of mono-quaternary ammonium compounds having the formula V:
R(CH₂)nN⁺R₁R₂R₃ X⁻ V
where R₁ is alkyl, R₂ is alkenyl or alkyl, R₃ is branched alkyl, alkenyl or (CH₂Y)s where s is 1-12 and Y is hydroxy or hydroxyphenyl, n is 6-16, R is H or phenyl, and X⁻ is halide, OTs⁻ or pharmaceutically acceptable salt thereof.

The fungal infections may also be treated with pharmaceutical compositions than include effective amounts of any one or more of a bis-quaternary ammonium compound having the formula VI:
R₁R₂R₃N⁺(CH₂)nN⁺R₁R₂R₃ mX⁻ VI
where R₁ and R₂ are independently CH₃ and C₂H₅; R₃ is C₁-C₁₁, alkyl, alkenyl or alkynyl, m is 1 or 2, n is 6-21; and X⁻ is halide, OTs—or a pharmaceutically acceptable salt thereof.

The disclosed compounds can be appropriated formulated for use in inhibiting fungal growth, such as inhibiting fungal growth on a surface, including skin or inorganic materials such as building materials, clothing and the like. Thus one may selected one or more of the alkyl mono- or bis-quaternary ammonium compounds disclosed or selected from the compounds of formula I. Suitable compositions may be applied by spraying or soaking the area, surface, material or object affected by the fungal presence or the fungal growth.

Human fungal infections as well as Trypanosomiasis or Leishmaniasis infections may be visceral, mucosal or cutaneous (skin) and can be treated topically or systemically by use of mono- or bis-alkyl ammonium compounds formulated for systemic or topical administration. 1,16-hexadecamethylene bis-[N-methylpyrrolidinium]dibromide (DTAB) is a preferred compound for use in such formulations.

Yet another aspect of the invention is a packaged formulation for use in treating fungal or Leishmaniasis infections. Such a package or kit may include pharmaceutical compositions, optionally in selected dosages, which contain a mono- or bis-alkyl ammonium compound selected from the disclosed compounds; e.g. of formula I-VI, and instructions for use.

Pharmaceutical Compositions

Pharmaceutical compositions and dosage forms of the invention comprise one or more active ingredients in relative amounts and formulated so that a given pharmaceutical composition or dosage form inhibits or cures fungal infections. Preferred pharmaceutical compositions and dosage forms comprise a compound of formula I or a pharmaceutically acceptable prodrug, salt, solvate or clathrate thereof, optionally in combination with one or more additional active agents.

Compositions containing the antifungal agent may be administered in several ways, including orally, parenterally, intraperitoneally, intradermally or intramuscularly. Pharmaceutical forms suitable for injection include sterile aqueous solutions or dispersions for extemporaneous preparation of the solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained by the use of a coating such as lecithin, by the maintenance of the required particle size in case of a dispersion and by the use of surfactants. The prevention of the action of microorganisms can be effected by various antibacterial and antifungal agents such as parabens, chlorobutanol, phenol, sorbic acid, thimerosal and the like. In many cases, isotonic agents may be included, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Oral dosage forms are also contemplated. Pharmaceutical compositions of the invention suitable for oral administration can be presented as discrete dosage forms, including but not limited to, tablets (e.g. chewable tablets), caplets, capsules and liquids such as flavored syrups. Dosage forms containing predetermined amounts of active ingredients may be prepared by well known methods of pharmacy, see Remington's Pharmaceutical Sciences (1990) 18th ed., Mack Publishing Co., Easton, Pa.

Typical oral dosage forms of the invention are prepared by combining the active ingredient(s) in an admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivates (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. One specific binder is a mixture of microcrystalline cellulose and sodium carboxymethyl cellulose sold as AVICEL RC-581. Suitable anhydrous or low moisture excipients or additives include AVICEL-PH-103J and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, preferable from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crosprovidone, polacrilin potassium, sodium starch glycolate, potato or tapioca starch, other starches, pre-gelatinized starch, other starches, clays, other algins, other cellulosses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

The pH of a pharmaceutical composition or dosage form, or of the tissue where the composition or dosage form is applied, may be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients to improve delivery. Stearates for example can serve as a lipid vehicle for the formulaltion, as an emulsifying agent or surfactant, and as a delivery-enhancing or penetration-enhancing agent. Salts, hydrates or solvates of the active ingredients can be used to further adjust the properties of the resulting compositions.

Upon formulation, solutions will be administered in a manner compatible with the dosage formulation and in such amount as is therapeutically effective. The formulations are easily administered in a variety of dosage forms preferably as injectable solutions.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intradermal and intraperitoneal administration. In this connection, sterile aqueous media that can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### DEFINITIONS

The term “alkyl” refers to the radical of saturated aliphatic groups, including straight-chain alkyl groups, and branched-chain alkyl groups. The term alkyl further includes alkyl groups, which can further include oxygen, nitrogen, sulfur or phosphorous atoms replacing one or more carbons of the hydrocarbon backbone, e.g., oxygen, nitrogen, sulfur or phosphorous atoms. In preferred embodiments, a straight chain or branched chain alkyl has 30 or fewer carbon atoms in its backbone (e.g., C₁-C₃₀ for straight chain, C₃-C₃₀ for branched chain), preferably 20 or fewer, and more preferably 18 or fewer.

Moreover, the term alkyl as used throughout the specification and claims is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate.

The term “alkyl” also includes unsaturated aliphatic groups analogous in length and possible substitution to the alkyls described above, but that contain at least one double or triple bond respectively. An “alkylaryl” moiety is an alkyl substituted with an aryl (e.g., phenylmethyl(benzyl)).

The terms “alkoxy,” “aminoalkyl” and “thioalkoxy” refer to alkyl groups, as described above, which further include oxygen, nitrogen or sulfur atoms replacing one or more carbons of the hydrocarbon backbone, e.g., oxygen, nitrogen or sulfur atoms.

The terms “alkenyl” and “alkynyl” refer to unsaturated aliphatic groups analogous in length and possible substitution to the alkyls described above, but that contain at least one double or triple bond, respectively. For example, the invention contemplates cyano and propargyl groups.

The term “aralkyl” means an aryl group that is attached to another group by a (C₁-C₆) alkylene group. Aralkyl groups may be optionally substituted, either on the aryl portion of the aralkyl group or on the alkylene portion of the aralkyl group, with one or more substituents.

The term “aryl” as used herein, refers to the radical of aryl groups, including 5- and 6-membered single-ring aromatic groups that may include from zero to four heteroatoms(heteroaryl), for example, benzene, pyrrole, furan, thiophene, imidazole, benzoxazole, benzothiazole, triazole, tetrazole, pyrazole, pyridine, pyrazine, pyridazine and pyrimidine, and the like. Aryl groups also include polycyclic fused aromatic groups such as naphthyl, quinolyl, indolyl, and the like.

Those aryl groups having heteroatoms in the ring structure may also be referred to as “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhlydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term “cyclyl” refers to a hydrocarbon 3-8 membered monocyclic or 7-14 membered bicyclic ring system having at least one non-aromatic ring, wherein the non-aromatic ring has some degree of unsaturation. Cyclyl groups may be optionally substituted with one or more substituents. In one embodiment, 0, 1, 2, 3, or 4 atoms of each ring of a cyclyl group may be substituted by a substituent. The term “cycloalkyl” refers to a hydrocarbon 3-8 membered monocyclic or 7-14 membered bicyclic ring system having at least one saturated ring. Cycloalkyl groups may be optionally substituted with one or more substituents. In one embodiment, 0, 1, 2, 3, or 4 atoms of each ring of a cycloalkyl group may be substituted by a substituent. Cycloalkyls can be further substituted, e.g., with the substituents described above. Preferred cyclyls and cycloalkyls have from 3-10 carbon atoms in their ring structure, and more preferably have 3, 4, 5, 6 or 7 carbons in the ring structure. Those cyclic groups having heteroatoms in the ring structure may also be referred to as “heterocyclyl,” “heterocycloalkyl” or “heteroaralkyl.” The aromatic ring can be substituted at one or more ring positions with such substituents as described above.

The terms “cyclyl” or “cycloalkyl” refer to the radical of two or more cyclic rings (e.g., cycloalkyls, cycloalkenyls, cycloalkynyls, aryls, heteroaryls, and/or heterocyclyls). In some cases, two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkyl, alkylaryl, or an aromatic or heteroaromatic moiety.

The term “haloalkyl” is intended to include alkyl groups as defined above that are mono-, di- or polysubstituted by halogen, e.g., fluoromethyl and trifluoromethyl.

The term “halogen” designates —F, —Cl, —Br or —I.

The term “hydroxyl” means —OH.

The term “heteroatom” as used herein means an atom of any element other than carbon or hydrogen. Preferred heteroatoms are nitrogen, oxygen, sulfur and phosphorus.

The term “mercapto” refers to a —SH group.

The term “sulfhydryl” or “thiol” means —SH.

Certain antifungal compounds may encompass various isomeric forms. Such isomers include, e.g., stereoisomers, e.g., chiral compounds, e.g., diastereomers and enantiomers.

The term “chiral” refers to molecules that have the property of non-superimposability of the mirror image partner, while the term “achiral” refers to molecules that are superimposable on their mirror image partner.

The term “diastereomers” refers to stereoisomers with two or more centers of dissymmetry and whose molecules are not mirror images of one another.

The term “enantiomers” refers to two stereoisomers of a compound, which are non-superimposable mirror images of one another. An equimolar mixture of two enantiomers is called a “racemic mixture” or a “racemate.”

The term “isomers” or “stereoisomers” refers to compounds that have identical chemical constitution, but differ with regard to the arrangement of the atoms or groups in space.

Furthermore the indication of configuration across a carbon-carbon double bond can be “Z” referring to what is often referred to as a “cis” (same side) conformation whereas “E” refers to what is often referred to as a “trans” (opposite side) conformation. Regardless, both configurations, cis/trans and/or Z/E are contemplated for the compounds for use in the present invention.

With respect to the nomenclature of a chiral center, the terms “d” and “l” configuration are as defined by the IUPAC Recommendations. As to the use of the terms, diastereomer, racemate, epimer and enantiomer, these will be used in their normal context to describe the stereochemistry of preparations.

Natural amino acids when used in association with the present invention are in the “l” configuration, unless otherwise designated. Unnatural or synthetic amino acids are in the “d” configuration, unless otherwise designated.

Radiolabels may be incorporated in any of the formulae delineated herein. Such compounds have one or more radioactive atoms (e.g., ³H, ²H, ¹⁴C, ¹³C, ³⁵S, ³²P, ¹²⁵I, ¹³¹I) introduced into the compound. Such compounds are useful for drug metabolism studies and diagnostics, as well as therapeutic applications.

The term “obtaining” as used is intended to include purchasing, synthesizing or otherwise acquiring the antifungal compounds.

The term “prodrug” includes compounds with moieties that can be metabolized in vivo. Generally, the prodrugs are metabolized in vivo by esterases or by other mechanisms to active drugs. Examples of prodrugs and their uses are well known in the art (see, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included.

### BRIEF DESCRIPTION OF THE FIGURES

FIG. 1. Effect of mono- and bis-quaternary ammonium compounds on the growth of S. cerevisiae as demonstrated by the IC₅₀ values. Wild-type yeast cells were inoculated at 10⁵ cells/ml in the presence of increasing concentrations of the compounds indicated.

FIG. 2. Effect of the size of the cationic head group of choline analogs on the growth of S. cerevisiae as demonstrated by the IC₅₀ values. Wild-type yeast cells were inoculated at 10⁵ cells/ml in the presence of increasing concentrations of the compounds indicated.

FIG. 3. Effect of the size of the lipophilic chain of choline analogs on the growth of S. cerevisiae as demonstrated by the IC₅₀ values. Wild-type yeast cells were inoculated at 10⁵ cells/ml in the presence of increasing concentrations of the compounds indicated.

FIG. 4. Growth inhibition of C. albicans by 10 μM of choline analogs.

FIG. 5. Phospholipid metabolism in S. cerevisiae and P. falciparum. Pathways for the synthesis of the major phospholipids in S. cerevisiae (gray thin arrows) and P. falciparum (black thick arrows). 1: phosphatidylserine synthase (Pss1), 2: phosphatidylserine decarboxylases (Psd1 and Psd2), 3: phosphatidylethanolamine methyltransferase (Pem1), 4: phospholipid methyltransferase (Pem2), 5: choline kinase (Cki1), 6: phosphocholine cytidylyltransferase (Pct1), 7: choline phosphotransferase (Cpt1), 8: ethanolamine kinase (Eki1), 9: phosphoethanolamine cytidylyltransferase (Ept1), 10: ethanolamine phosphotransferase (Ect1), 11: phosphoethanolamine methyltransferase (PfPmt). PA: phosphatidic acid; CDP-DAG: cytidylphosphate diacylglycerol; PtdSer: phosphatidylserine; PtdEtn: phosphatidylethanolamine; PME: phosphatidylmonomethylethanolamine; PDE: phosphatidyldimethylethanolamine; PtdCho: phosphatidylcholine; PtdIno: phosphatidylinositol.

FIG. 6A. Chemical structure of G25 (1,16-hexadecamethylenebis[N-methylpyrrolidinium]dibromide) and T16 (1,12-dodecanemethylene bis[4-methyl-5-ethylthiazolium]diodide).

FIG. 6B. Inhibition of S. cerevisiae growth by G25 and its analog T16. Liquid growth assays were performed in increasing concentrations of G25 and T16 as described.

FIG. 7. Inhibition of choline uptake in S. cerevisiae by G25. Choline transport in the wild-type strain of S. cerevisiae was measured as described in Materials and Methods. The uptake of 1 μM [methyl-³H]-choline in the presence of 100 μM ethanolamine (Etn), 4-, 20- and 100-fold excess of cold choline, G25 and T16 is shown as a percent of the counts obtained in the control (Ctrl: without drugs, choline or ethanolamine).

FIG. 8A. Transport kinetics of a radiolabeled G25 analog, [³H]-T16, in S. cerevisiae. Transport of [³H]-T16 was measured as described in Materials and Methods in nitrogen-free medium. Wild-type (black and white circles) and hnm1□ (black and white triangles) strains were assayed for uptake of [³ H]-T16 overtime at 30° C. (black circles and triangles) and 4° C. (white circles and triangles). Each value is the mean ± standard deviation of triplicate determinations of a typical experiment.

FIG. 8B. Transport kinetics of a radiolabeled G25 analog, [³H]-T16, in wild-type S. cerevisiae as a function of the concentration of T16. Transport of the indicated concentrations of [³H]-T16 in wild-type S cerevisiae was measured at 30° C. for 4 min. The curve was fitted to the Michaelis-Menten equation (Vₘₐₓ×S[Kₘ+S]). The Lineweaver-Burk representation of the saturation curve is shown as an inset. Only one representative experiment performed in triplicate from two independent experiments is shown.

FIG. 8C. Transport kinetics of a radiolabeled G25 analog, [³H]-T16, in hnm1□ as a function of the concentration of T-16. Transport of the indicated concentrations of [³H]-T16 in the hnm1□ strain was measured at 30° C. for 4 min. The curve was fitted to the Michaelis-Menten equation (Vₘₐₓ×S[Kₘ+S]). The Lineweaver-Burk representation of the saturation curve is shown as an inset. Only one representative experiment performed in triplicate from two independent experiments is shown.

FIG. 9. Sensitivity to G25 of wild type and mutants affected in different steps of PtdCho biosynthesis. Plate growth limiting dilution assays were performed as described in Materials and Methods in the absence or the presence of 5 μM of G25. The strains and genes deleted in the strains used are described in “Material and Methods”.

FIG. 10A. Effect of G25 on PtdEtn and PtdCho synthesis from choline. 5-6×10⁷ synchronized P. falciparum-infected erythrocytes (7% trophozoite stage) were pre-incubated at 4% haematocrit for 1 h in RPMI-based medium containing the indicated concentration of G25 before adding 30 μM [methyl-³H]-choline (334 mCi/mmol). After incubation at 37° C. for 3 h, the cellular lipids were extracted and fractionated on TLC plates for quantification of radioactivity in PtdCho (black squares). Each value is the mean±standard deviation of triplicate determinations of two independent experiments.

FIG. 10B. Effect of G25 on PtdEtn and PtdSer synthesis from serine. 5-6×10⁷ synchronized P. falciparum-infected erythrocytes (7% trophozoite stage) were pre-incubated at 4% haematocrit for 1 h in RPMI-based medium containing the indicated concentration of G25 before adding 10 μM [¹⁴C]-serine (57 mCi/mmol). After incubation at 37° C. for 3 h, the cellular lipids were extracted and fractionated on TLC plates for quantification of radioactivity in PtdSer (black diamonds), and PtdEtn (white circles). Each value is the mean±standard deviation of triplicate determinations of two independent experiments.

FIG. 11A. Effect of G25 on the activity of purified recombinant PfPsd1 enzyme. PfPsd1 activity was determined as described in Materials and Methods by measuring the amount of [¹⁴C]-PtdEtn formed from PtdSer. TLC analysis of PfPsd1-mediated conversion of PtdSer into PtdEtn in the absence and presence of increasing concentrations of G25 was performed.

FIG. 11B. Quantitative analysis of the TLC data shown in FIG. 11A. Values are means±standard deviation of triplicate determination of two independent experiments.

### DETAILED DESCRIPTION OF THE INVENTION

The invention had its genesis in a series of studies on the effect of quaternary ammonium compounds on the nonpathogenic fungus Saccharomyces cerevisiae, the fungal pathogen Candida albicans and the agent of human cutanuous leishmaniasis, Leishmania major. Initially, the goal was to understand the mechanism of action of this class of compounds against the human malaria parasite Plasmodium falciparum because the structural similarity of these compounds to choline suggested that they might act by blocking choline transport into the parasite. It was reasoned that because Saccharomyces cerevisiae, Candida albicans and Leishmania major grow in the absence of choline, the quaternary compounds would have no effect on fungal or Leishmanial growth.

Surprisingly, several quaternary ammonium compounds exhibited very potent antifungal and anti-Leishmanial activities in vitro.

History of the Development of Quaternary Ammonium Choline Analogs as Antimalarials

Plasmodium falciparum, the causative agent of the most severe form of human malaria, is responsible for over 2 million deaths annually (WHO, 2000). The emergence of drug-resistant parasites to the most commonly used antimalarials, such as chloroquine, mefloquine and pyrimethamine has hampered efforts to combat this disease, thus emphasizing the need to develop new compounds for malaria treatment and prophylaxis.

The rapid multiplication of P. falciparum in human erythrocytes requires active synthesis of new membranes. Therefore developing drugs that target membrane biogenesis was an attractive strategy to fight malaria. The finding that quaternary ammonium choline analogs inhibit the synthesis of new membranes and block the growth of the parasite has stimulated efforts to develop this class of compounds for anti-malarial chemotherapy (Ancelin, et al., 1985). Using a combinatorial chemistry approach to obtain compounds with greater specificity and potency against malaria, more than 420 choline analogs have been synthesized and their structure optimized using quantitative structural-activity criteria (QSAR) (Wengelnik, et al., 2002). These compounds displayed a very close correlation between the inhibition of parasite growth in vitro and specific inhibition of parasite membrane biogenesis (Ancelin, 1998).

One of these compounds, G25, inhibited P. falciparum growth in vitro and cleared malaria infection in monkeys infected with P. falciparum and P. cynomolgi at very low doses (Wengelnik, et al., 2002). A tritium-labeled bisquaternary ammonium salt analog of G25, VB5-T (IC₅₀˜18 nM), was shown to accumulate by several hundred-fold in trophozoite-infected compared to uninfected red blood cells (Wengelnik, et al., 2002). Accumulation of this agent within the parasite is linear with concentrations up to 1000-fold above its IC₅₀ and appears irreversible (Wengelnik, et al., 2002). The antimalarial potency of G25 is similar to chloroquine, which kills the parasite at low nanomolar extracellular concentrations but accumulates within the parasite food vacuole to millimolar range (Sullivan, et al., 1996). Although choline analogs are highly effective against malaria and are entering clinical evaluation, the difficulties in the experimental manipulation of P. falciparum has hampered efforts to understand their mode of action and identify their cellular targets.

Yeast Studies

The amenability of the yeast Saccharomyces cerevisiae to genetic manipulation has made it an invaluable system to characterize the metabolic pathways involved in the synthesis of phospholipids, sterols and fatty acids. The lipid composition of the S. cerevisiae's membranes consists largely of phosphatidylcholine (PtdCho) (44%), phosphatidylethanolamine (PtdEtn) (18%) and phosphatidylinositol (Ptdlno) (19.5%) (Jakovcic, et al., 1971). These glycerolipids are thought to be essential for S. cerevisiae growth in medium that contains glucose or nonfermentable carbon sources (Martin, 1969).

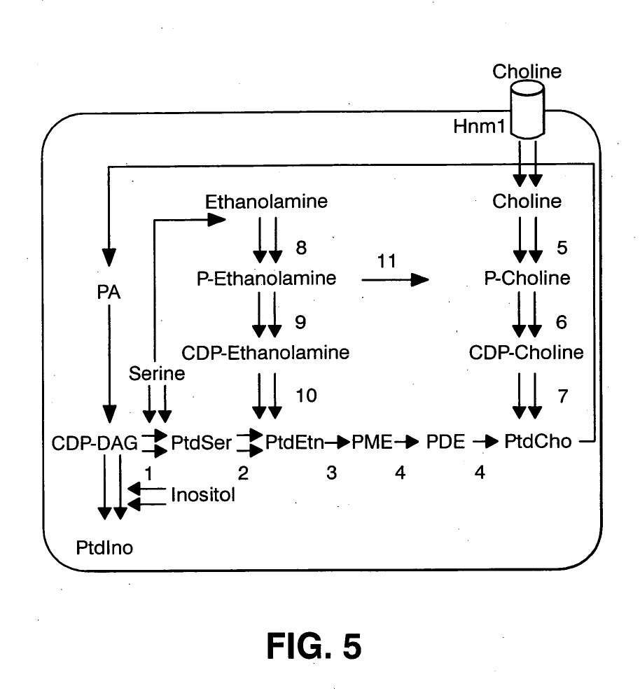

As summarized in FIG. 5, glycerolipid synthesis involves distinct but highly co-regulated biosynthetic pathways: (i) the CDP-choline pathway, which uses choline as a precursor for the de novo synthesis of PtdCho (Hjelmstad and Bell, 1987); (ii) the CDP-ethanolamine pathway, which uses ethanolamine as a precursor for the de novo synthesis of PtdEtn (Hjelmstad and Bell, 1991), (iii) the CDP-DAG pathway, which utilizes serine and CDP-DAG to form PtdSer, which is then decarboxylated to form PtdEtn, and (iv) the PtdIno pathway, which synthesizes PtdIno from CDP-DAG and inositol (Clancy, et al., 1993). The CDP-DAG and the CDP-ethanolamine pathways converge into PtdEtn, which is subsequently methylated in a three step AdoMet-dependent methylation to form PtdCho. This reaction is catalyzed by two methyl transferases encoded by the PtdEtn N-methyltransferase PEM1 and phospholipid N-methyltransferase PEM2 genes. The CDP-DAG pathway is the major pathway leading to the formation of PtdCho in S. cerevisiae (Carman and Henry, 1999) Therefore, in this organism, neither choline nor the enzymes of the CDP-choline pathway are essential for survival. The CDP-choline pathway becomes essential when the genes encoding the enzymes in the CDP-DAG pathway are altered or deleted (Kodaki and Yamashita, 1989).

Biochemical studies in P. falciparum and the available genome sequences have made it possible to define the pathways for synthesis of the major phospholipids (Gardner, et al., 2002) (FIG. 5). With the exception of the choline transporter and the phospholipid methyltransferases, all the genes encoding enzymes of the CDP-choline, CDP-ethanolamine and CDP-DAG pathways have been identified.

The similarity between P. falciparum and S. cerevisiae in the biogenesis of the major phospholipids suggested that yeast could be used as a surrogate system to characterize the function of P. falciparum phospholipid synthesizing genes and determine the mode of entry and cellular targets of antimalarial lipid inhibitors.

Unexpectedly, based on the biogenesis studies in yeast, the antimalarial choline analog G25 inhibited the growth of S. cerevisiae in vitro; moreover, in the same range of concentration used to inhibit malarial growth, it was an effective inhibitor of choline transport in wild-type yeast.

Similar initial rate and overall uptake of a radiolabeled bisquaternary ammonium analog of G25 was measured in both wild-type and hnm1Δ cells, lacking the only yeast choline transporter, Hnm1. These results demonstrated that the choline carrier Hnm1 does not mediate the entry of bis-quaternary ammonium compounds. Of eleven individual yeast knockouts lacking genes involved in different steps of PtdCho biosynthesis, four mutants altered in the de novo CDP-choline pathway, and one mutant lacking the PtdSer decarboxylase-encoding gene, PSD1, were highly resistant to G25.

The labeling studies in P. falciparum demonstrated that G25 completely and specifically inhibited the de novo CDP-choline-dependent PtdCho biosynthetic pathway. Surprisingly, higher concentrations of this compound resulted in the inhibition of synthesis of PtdEtn from PtdSer, but had no effect on any other step of the CDP-DAG pathway. Interestingly, it was found that G25 inhibits the PtdSer decarboxylase activity of purified recombinant PfPsd1 in a way similar to the inhibition of the native enzyme. Together these data indicate that G25 specifically targets the pathways for synthesis of the two major phospholipids phosphoatidylcholine and phosphatidylethanolamine to exert its antimalarial activity. These novel findings constituted important information for quaternary ammonium compounds that are under consideration for clinical studies, whether for use as antimalarials or as antifungal compounds based on these studies in yeast.

History of Identification of Antimalarial choline analogs

The first indication that quaternary ammonium compounds act as inhibitors of choline transport in red cells came from studies by Martin (1969). Vial, et al. (1984) showed that phospholipid metabolism, an important process for generation of new membranes following parasite multiplication, constitutes an effective target for antimalarial chemotherapy.

Choline and the ethanolamine analogs, 1-aziridine ethanol, dl-2-amino-1,3-propranediol and D-or L-2-amino-1-butanol, inhibit Plasmodium proliferation with an IC₅₀ of 50-80 μM. The Vial, et al. studies revealed that incorporation of analogs, in place of the natural polar head groups, into cellular phospholipids and/or modification of phospholipid composition, are deleterious to the growth of Plasmodium.

Further research showed that analogs of choline containing one or two quaternary ammonia groups; i.e., decyltrimethylammonium (DTMA), decamethonium (DMA) and hemicholinium 3 (HC3) are lethal to P. falciparum in vitro in a dose-dependent manner with IC₅₀ values of 0.7 μM, 1 μM and 4 μM, respectively (Ancelin and Vial, 1986). By increasing the length of the alkyl chain of decyltrimethylammonium by successive additions of two carbon atoms up to hexadecyltrimethylammonium, a decrease in the IC₅₀ values measured in P. falciparum growth assays has been observed (Ancelin, et al., 1998, 1996, 1985 and 1984).

These results were the starting point for a rational design approach by combinatorial chemistry to obtain compounds with greater specificity and potency (Calais, et al., 2000; 1997) as potential antimalarial drugs. More than 420 choline analogs were synthesized and their structure optimized using quantitative structural-activity criteria (QSAR) (Vial, 2001). The compounds were optimized for in vitro antimalarial activity, and displayed a very close correlation between the inhibition of parasite growth in vitro and specific inhibition of parasite phospholipid biosynthesis (Wengelnik, et al., 2002).

First generation compounds contained a duplication of the cationic group (bis-quaternary ammonium salts, volume ˜400-600 A³) separated by a long lipophilic alkyl chain (n≧12 methylene groups (>17 A)). One of those compounds, G25 [1,16-hexadecamethyenebis (N-methypyrrolidinium) dibromide](IC₅₀=1.2 nM), was effective against multidrug resistant laboratory and clinical isolates of P. falciparum with lC₉₀/IC₅₀ ratios of less than 3, indicating inhibition of a specific target and not a general cytotoxic effect (Wengelnik, et al., 2002).

To optimize absorption and diffusion into tissues, second generation analogs were synthesized. These drugs contained two basic head groups (pKa>9) separated by a lipophilic spacer. The basic head groups are protonated at a physiological pH (amine, amidine and guanidine function), thus mimicking the cationic head of choline. The equilibrium between the protonated and unprotonated forms increases the degree of diffusion of these compounds into tissues. Modification of a quaternary ammonium to a tertiary amine results in a 100-fold decrease in IC₅₀ values (Vial, 2001).

Introduction of an amidine or guanidine was critical for generation of very potent antimalarial compounds with IC₅₀ values as low as 0.1 pM. Three compounds, MS1 (aromatic amidine in which the amidine function is present in the 1,2-dihydropyridine group), M53 and M60 (amidine function not conjugated, and N-atoms substituted by R alky groups to modulate the lipophilicity of the molecule, with optimal availability) were chosen as lead compounds. To enhance oral bio-availability of the compounds, nonionic pro-drugs were synthesized. Third generation lead compounds were thioester or disulfide prodrugs with IC₅₀˜0.5-2 nM.

Based on the unexpected susceptibility of Candida albicans and S. cerevisiae to the known antimalarial ammonium choline analogs, the inventor tested these antimalarial compounds to determine potential candidate compounds for development as antifungal agents. Several of the alkyl ammonium choline analogs exhibited excellent IC50s in in vitro tests and indicated several activity-structure relationships contributing to inhibition of fungal growth.

The following examples are intended to illustrate the invention and/or background considered in the development of the invention. The examples are not intended to be limiting and while the description has been described with respect to preferred embodiments, those skilled in the art will readily appreciate that various changes and/or modifications can be made to the invention without departing from the spirit or scope of the invention, either in the description or in the appended claims.

### EXAMPLES

### MATERIALS AND METHODS

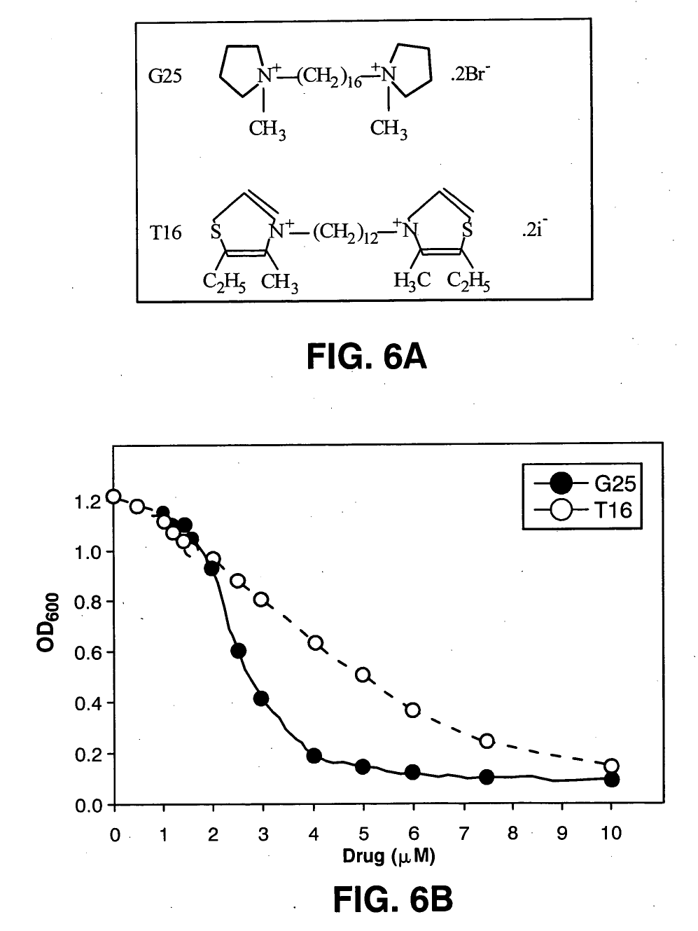

Chemicals: G25 (1,16-hexadecamethylenebis[N-methylpyrrolidinium]dibromide) (Calas, et al., 2000) T16 (1, 12-dodecanemethylene bis[4-methyl-5-ethylthiazolium]diiodide) and [³H]-T16 were synthesized by conventional procedures (FIG. 6A and 6B).

Strains and Growth Conditions: Wild-type (BY4741: Mata his3Δ1 leu2Δ0 met15Δ0 ura3Δ0) and mutant (hnm1Δ, psd1Δ, cki1Δ, pem1Δ, ept1Δ, cpt1Δ, eki1Δ, psd2Δ, pct1Δ, ect1Δ, pem2Δ) S. cerevisiae strains used in this study were purchased from Research Genetics (Invitrogen, USA). These strains were grown on YPD (1% yeast extract, 2% dextrose and 2% peptone) or synthetic complete medium (SD, 1.7% yeast nitrogen base, 5% ammonium sulfate and 2% dextrose). The Nigerian strain of P. falciparum was propagated in human red blood cells at 4% hematocrit by the method of Trager and Jensen (1976). Plate growth assays were performed by growing wild-type and mutant yeast strains in YPD to mid-log phase. Cultures were serially diluted 1:10 starting with a density of 3×10⁷ cells/ml. The growth of cells was monitored by spotting 3 μl of each dilution onto solid medium in the absence or presence of 5 μM of G25. Growth assays in liquid media were performed by inoculating wild-type and hnm1Δ cells to a density of 1×10⁴ cells/ml in YPD supplemented with increasing concentrations of choline analogs. The OD₆₀₀ was measured when the control without choline analogs reached a density of 1.8×10⁷ cells/ml.

Uptake Assays: Yeast strains were grown in synthetic complete medium supplemented as required to maintain cell growth to an optical density of 0.55-0.65 at 600 nm. Cells were harvested by centrifugation at 3,200×g for 10 min at 4° C., washed twice in cold PBS and resuspended in nitrogen-free medium (SD without ammonium sulfate). Each reaction was performed in a 1 ml final volume in the presence of 12 nM of [methyl-³H]-choline (82 Ci/mmol; Amersham). After 3 min incubation at 30° C. with shaking, transport was immediately stopped by filtration through Whatman GF/C glass microfiber paper. The filters were washed three times with 5 ml ice-cold PBS, air dried, and analyzed in a scintillation counter. For time course uptake, 3-4×10⁷ cells were incubated at 30° C. in 1 ml of nitrogen-free medium in the presence of 25 nM [³H]-T16 (69 Ci/mmol) for different time periods (1, 2, 3, 4, 5, 7, 10 and 15 min) after which 5 ml of cold PBS was added to stop the reaction. Kinetic parameters were determined after 4 min of incubation at 30° C. in the presence of [³H]-T16 at concentrations ranging from 25 nM to 75 μM (69 Ci - 23 mCi/mol). The samples were centrifuged at 4° C. for 10 min at 1,200×g, the supernatants were discarded and the cells were then resuspended in 5 ml of cold PBS. The reaction was terminated by filtering the cell suspension through GF/C membranes that were pretreated with 15 ml of 0.05% polyethyleneimine (PEI). The filters were washed twice with 5 ml of cold PBS, air dried, and analyzed in a scintillation counter. The cellular accumulation ratio (CAR) was calculated as previously described for T16 and G25 (Biagini, et al., 2003; Wengelnik, et al., 2002).

Labeling Studies and Phospholipid Analysis in P. falciparum: Nigerian strains of P. falciparum were asexually cultured in the presence of basic medium (RPMI 1640 supplemented with 25 mM Hepes, pH 7.4) and 10% AB⁺ human serum (Trager and Jensen, 1976). Parasite synchronization was obtained with three successive 5% sorbitol treatments (Lambros and Vanderberg, 1979). Synchronized P. falciparum-infected erythrocytes (7-10% parasitemia, trophozoites) were pre-incubated for 1 hour at 37° C. at 4% hematocrit in 2 ml (final volume) basic medium in the absence or the presence of different concentrations of the compound G25. The appropriate radioactive precursor of lipid metabolism was added followed by further 3 h of incubation. Radioactive precursors were used as follows: 30 μM [methyl-³H]-choline (334 mCi/mmol), 2 μM [³H]-ethanolamine (2 Ci/mmol) and 10 μM [3-¹⁴C]-serine (57 mCi/mmol). Following incubation with radiolabeled precursors, cells were concentrated by centrifugation at 1,200×g for 5 min at 4° C., washed twice and the cellular lipids were extracted by a mixture of chloroform/methanol (Folch, et al., 1957) and the organic phase was evaporated under air. The dried material was dissolved in 100 μl of chloroform-methanol (9:1, v/v), and lipids were separated by Thin Layer Chromatography. Samples were applied to pre-coated silica gel plates (Merck, Darmstadt, Germany), which were developed in chloroform-methanol-acetic acid-sodium borate 0.1M (75:45:12:3. v/v/v/v). Phospholipids spots were revealed with iodine vapors and identified using appropriate standards. The silica gel of the lipid spots were scraped directly into scintillation vials containing 3 ml of liquid-scintillation fluid and counted in a Beckman LS 5000 spectrophotometer. The amount of labeled precursors incorporated into cellular lipids (nmol×10⁷ cell⁻¹×h⁻¹) was computed on the basis of radioactivity incorporated into lipids and the specific activity of the precursors in the incubation medium.

PtdSer Decarboxylase Assay

Recombinant P. falciparum PtdSer decarboxylase was purified as described by Baunaure and colleagues (Baunaure, et al., 2004) The assay mixture (0.3 ml) contained 0.1 M potassium phosphate buffer (pH 6.8), 0.06% Triton X-100 (w/v), 200 μM L-[dipalmitoyl]phosphatidyl[3-¹⁴C]-serine (1.35 mCi/mmol; Amersham), and the enzyme fraction containing recombinant protein of P. falciparum (240 μg). After incubation at 37° C. for 1 hour, the reaction was terminated by the addition of 400 μl chloroform. Chloroform-soluble materials were extracted, dried, and then dissolved in chloroform/methanol (9:1, v/v). Phospholipids were separated by Thin Layer Chromatography as described above. The radioactive phospholipids were localized and identified using appropriate standards, and radioactivity was quantified using the phosphoimager analyzer (Molecular Dynamics).

### Example 1

### Inhibition of Growth of S. cerevisiae by Choline Analogs

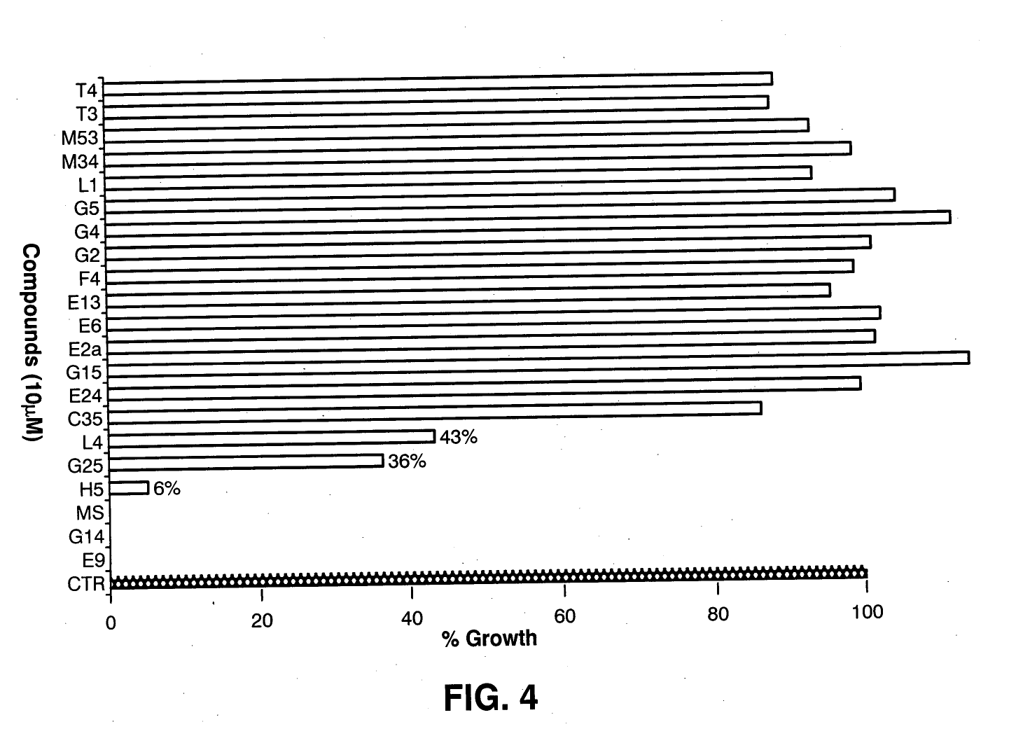

To assess the effect of choline analogs on the in vitro growth of S. cerevisiae in vitro, wild-type strains W303 and BY4741 were inoculated at 10⁵ cells/ml in a rich medium (YPD containing yeast extracts, peptone and glucose) in the presence of increasing concentrations of various choline analogs and incubated at 30° C. for 24 h. Growth inhibition was assessed by measuring the OD₆₀₀ and comparing it to that of the wild-type strain grown under the same conditions in absence of choline analogs. Twenty-one analogs including first, second and third generation (E2a, E6, E9, E13, E24, F4, G2, G4, G5, G14, GI5, G25, H5, L1, L4, M34, M53, MS1, T3 and T4) compounds (provided by Dr. Henri Vial, City, Country) were tested (FIG. 4). The choice of the compounds was such that they represent all possible structural features introduced during the optimization process for enhancing antimalarial activity as determined using the combinatorial chemistry techniques.

### Example 2

### Role of Duplication of the Polar Head Group

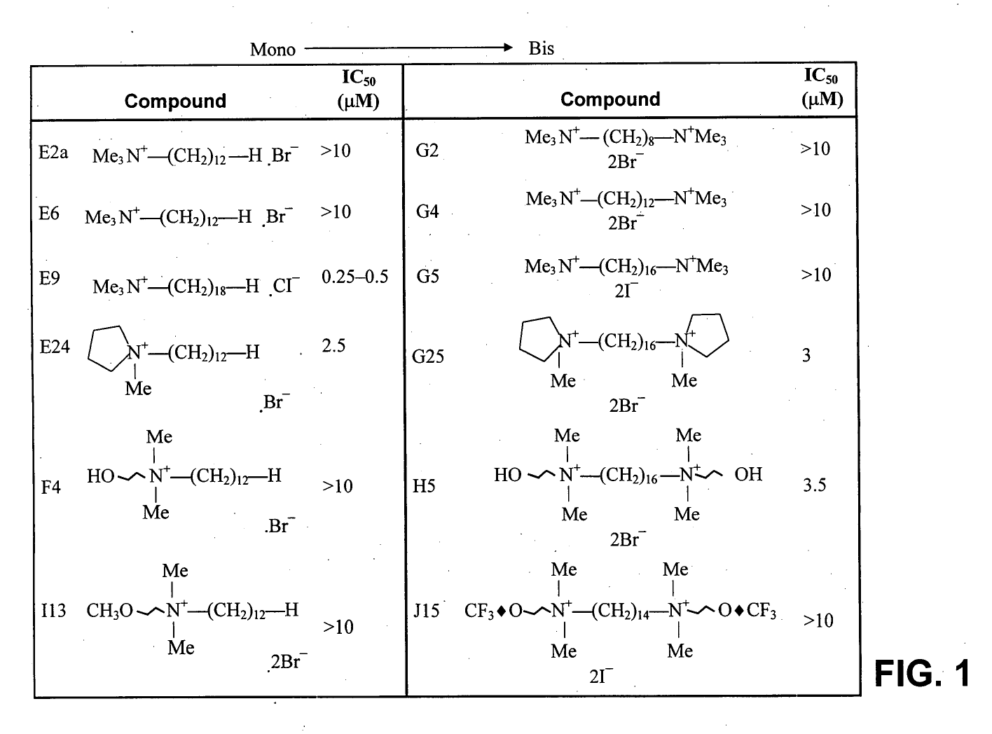

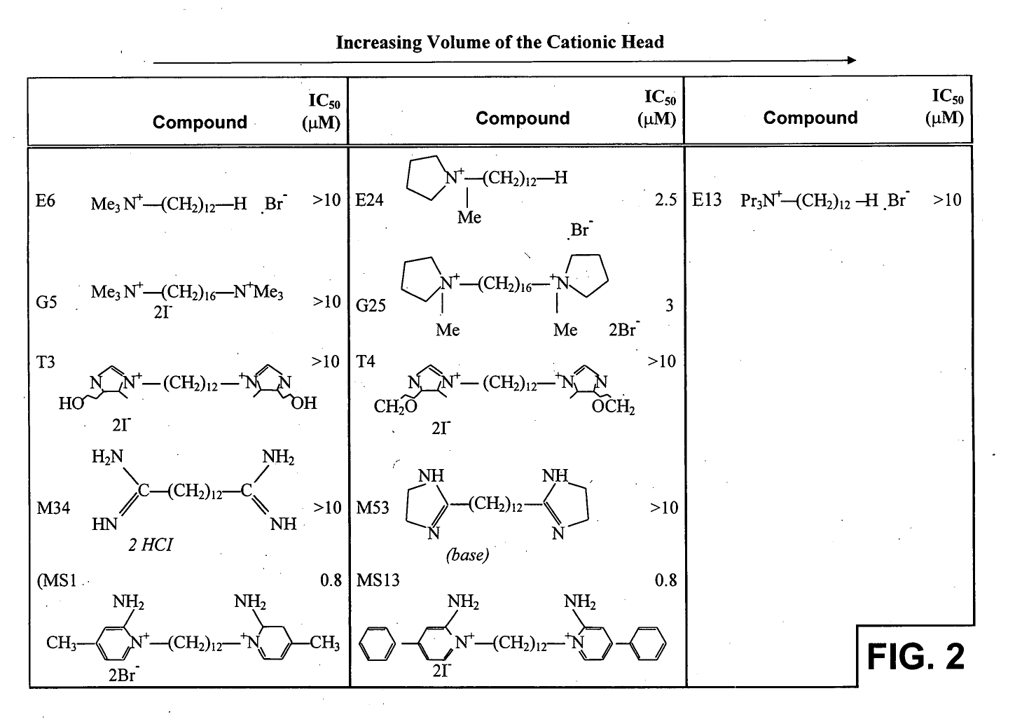

Mono- (E2a, E6, E9, E24, F4 and 113 compounds) and Bis(G2, G4, G5, G25, H5 and J15 compounds) quaternary ammonium compounds (FIG. 1) were used to test any possible correlations between duplication of the polar head group and the anti-fungal properties of the compounds. As shown in FIG. 2, only E9, G25 and E24 inhibited S cerevisiae growth. Duplication of the polar head group of E9 as in E24 resulted in a complete loss of activity. This suggested that compounds with a single quaternary ammonium group are more effective in yeast. Although duplication of the N,N-dimethyl group in F4 resulted in a compound, H5, with better efficacy, the alkyl chain in H5 is longer and it is likely that combination of both effects resulted in better potency. No major difference in IC₅₀ values was observed between E24 and G25, although the latter possesses a duplication of the head group containing a cyclic tetramethylene. These findings suggest that when the polymethylene chain contains more than 12 methylene groups, the monoquaternary ammonium compounds are more effective than the corresponding bis-quaternary compounds. An exception is if a cyclic tetramethylene substitutes for two methyl groups, as in G25.

### Example 3

### Role of the Bulk of the Cationic Head

The importance of the volume of the polar head group was determined by comparing the effect of E6, G5, T3, M34, MS 1 compounds and that of their derivatives E24/E 13, G25, T4, M53 and MS13, respectively (FIG. 2). Substitution of two of the methyl groups of E6 by a cyclic tetramethylene (E24) resulted in an improved potency of the compound. Similar results were seen when the cyclic tetramethylene group was duplicated as in G25. However, when the three methyl groups of E6 were substituted by three propyl groups (E13) no improvement was detected. It seems from these results that the volume of the cationic head does not affect the potency of the compound, which may relate more to the nature of the substitution.

### Example 4

### Role of the Length of the Lipophilic Chain

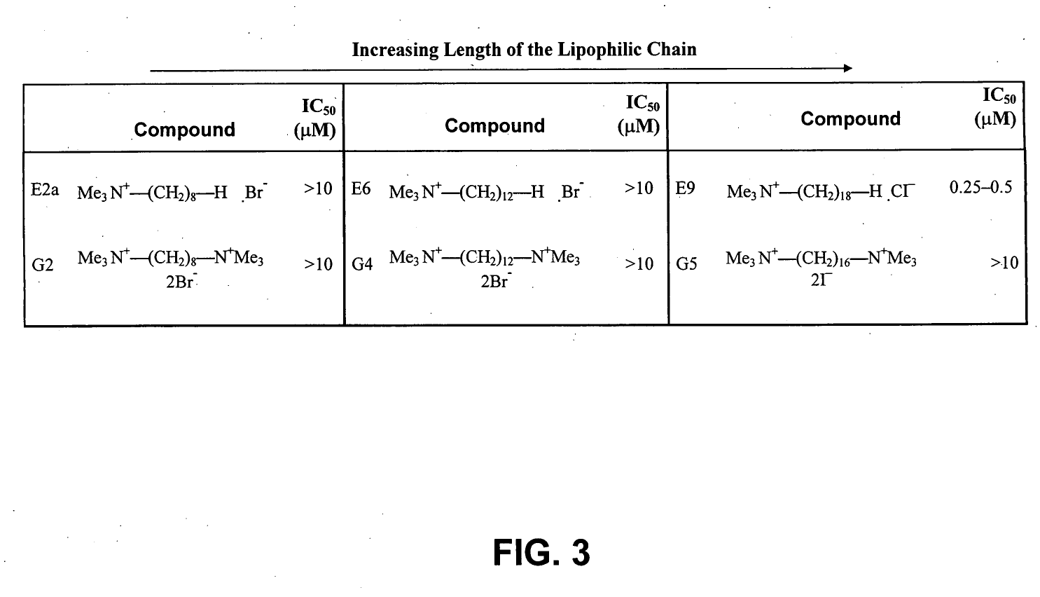

The length of the lipophilic chain was assessed by comparing the effect of E2a and G2 compounds, which contain an alkyl chain with 8 carbon atoms, with that of compounds E6 (C12)/E9 (C18) and G4 (C12)/G5 (C16) containing similar head groups but with various lengths of the alkyl chain (FIG. 3). As in P. falciparum, compounds with an alkyl chain of less than 12 carbon atoms were ineffective. Increasing the length from C12 in E6 to C18 in E9 resulted in a dramatic decrease of the IC₅₀ from high μM range ≈0.25-0.5 μM. Although E6 is not an effective drug, it is more effective than E2a. The presence of a duplication of the quaternary ammonium head group resulted in a complete loss of activity, independent of the size of the alkyl chain.

### Example 5

### Effect of Choline Analogs on Candida albicans

Candida albicans is able to cause recalcitrant infections. Infection by this organism can be superficial or hematogenously disseminated, resulting in life threatening systemic candidiasis. In this example the possible use of antimalarial choline analogs as antifungal agents was assessed.

The 23 choline analog compounds described above for their inhibitory effect on the growth of the C. albicans strain BWP17 (CA14 derivative) were tested in vitro. Six compounds (E9 (IC₅₀˜1 μM), G14 (IC₅₀˜0.5 pM), G25 (IC₅₀˜5 μM), H5 (IC₅₀˜5 μM), L4 (IC₅₀˜5 μM) and MS 1 (IC₅₀˜2.5 μM)) among the nine found to inhibit S. cerevisiae were also found to inhibit C. albicans proliferation (FIG. 4). G15, E24 and C35 inhibited at higher concentrations (>10 μM). Choline transport assays show that these compounds compete for choline entry into cells.

Although these compounds have been optimized previously for antimalarial potency, the results clearly suggest the use of E9 and G14 as lead compounds for development of effective antifungal drugs.

### Example 6

### Choline Analogs Inhibit Growth of Leishmania major

Choline analogs were also tested as growth inhibitors of the promastigote form of L. major. Of the 23 compounds tested, 13 showed an inhibitory effect at doses lower than 10 μM (Table 1). For those drugs, IC₅₀ values were determined. The most effective drugs were E9, E13, C35, E24, MS1, and G15 in the μM range, followed by E6, F4, G14, G25 and T4 in the low μM range.

IC₅₀ of Choline Analogs in Leishmania major.

TABLE 1

| | drug | | | | | | | | | | | | |
|---|---|---|---|---|---|---|---|---|---|---|---|---|---|
| | C35 | E6 | E9 | E13 | E24 | F4 | G14 | G15 | G25 | L4 | M34 | MS1 | T4 |
| WT* | 0.45 | 2.15 | 0.11 | 0.35 | 0.70 | 4.1 | 1.2 | 0.8 | 8.1 | 1.2 | 3.57 | 0.43 | 8.1 |

*The values are indicated in μM.

### RESULTS

The antimalarial drug G25 inhibits the growth of Saccharomyces cerevisiae. To examine the effect of the antimalarial choline analog G25 (1,16-hexadecamethylenebis(N-methylpyrrolidinium)dibromide) (FIG. 6A) on the growth of S. cerevisiae in vitro, wild-type strain BY4741 was inoculated at 1×10⁴ cells/ml in liquid medium in the presence of increasing concentrations of the compound and incubated at 30° C. for 16 h. Growth inhibition was assessed by measuring the OD₆₀₀ and comparing it to that of the wild-type strain grown under the same conditions in the absence of the compound. G25 inhibited yeast growth with an IC₅₀ of 2.5 μM (FIG. 6B). This IC₅₀ value is in the range of the predicted intracellular concentration of G25 in P. falciparum due to the accumulative properties inside the infected erythrocytes.

Uptake analysis and inhibition of choline transport by choline analogs in S. cerevisiae.

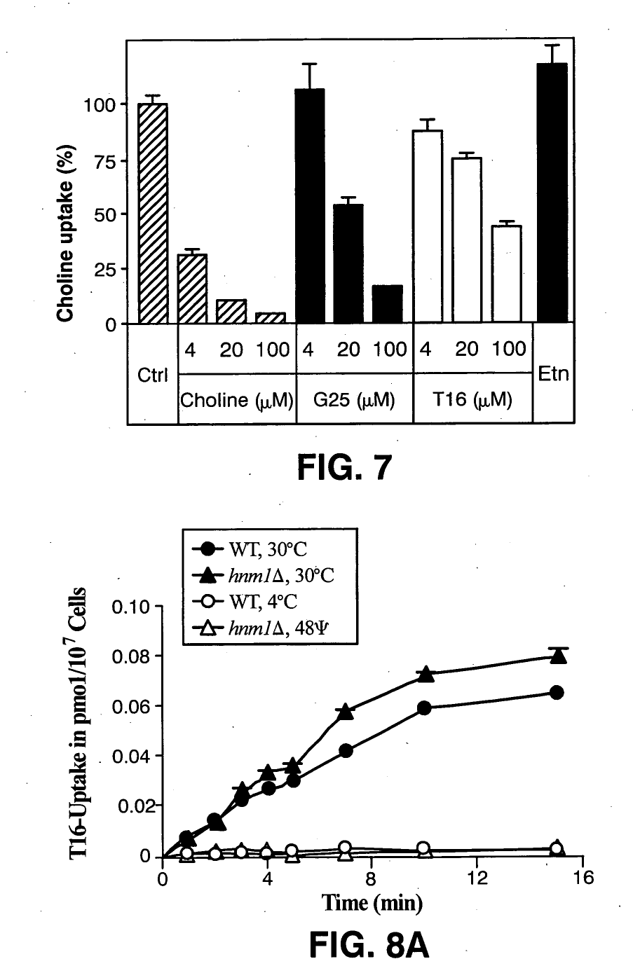

Bisquaternary ammonium choline analogs have been shown to inhibit choline entry into Plasmodium-infected erythrocytes. To determine whether these compound block choline uptake in S. cerevisiae, the transport of [methyl-³ H]-choline was examined in the absence or presence of various concentrations of G25 and its structural analog T16 (1,12-dodecanemethylene bis[4-methyl-5-ethylthiazolium]diiodide) (FIG. 6A) predicted by QSAR studies and confirmed experimentally to have potent and similar in vitro anti-malarial and anti-fungal inhibitory activities as G25 with IC₅₀ values of ˜16 nM and ˜4 μM (FIG. 6B), respectively. G25 inhibited choline uptake in a dose-dependent manner with 50% inhibition of choline transport in 20-fold excess and 84% inhibition in 100-fold excess (FIG. 7). The G25 analog, T16, also inhibited choline transport, albeit less efficiently, as G25 with 26% inhibition of choline transport in 20-fold excess and 57% inhibition in 100-fold excess (FIG. 7). As a control, 20- and 100-fold excess of unlabeled choline inhibited uptake of radiolabeled choline by 89 and 97%, respectively. Altogether, these data suggest that bisquaternary ammonium compounds are excellent inhibitors of choline uptake in S. cerevisiae.

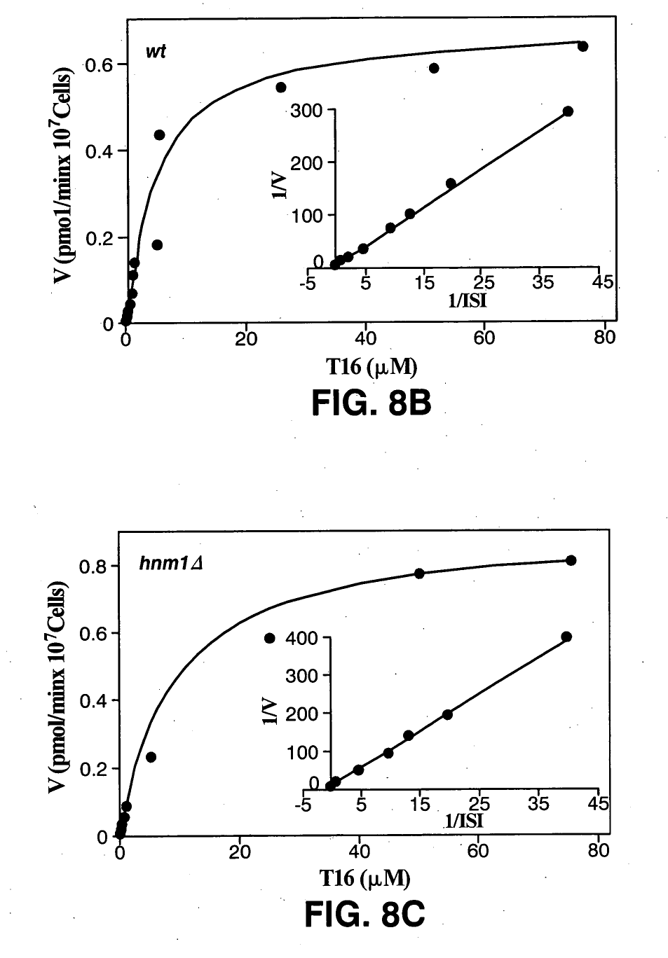

To directly examine the transport of choline analogs in S. cerevisiae, a tritium-labeled bisquaternary ammonium salt, [³H]-T16 was synthesized and examined with respect to its transport properties in wild-type cells at 4° C. and 30° C. No significant uptake of T16 in yeast could be measured at 4° C. (FIG. 8A). In contrast, [³H]-T16 uptake could be measured at 30 ° C. and was linear during the first 12 min after which it reached a plateau suggesting that entry of bisquaternary ammonium compounds into yeast cells is carrier mediated (FIG. 8A). Unlike P. falciparum-infected erythrocytes where G25 and T16 have been shown to accumulate with cellular accumulation ratios (CAR) after 3 h incubation of ˜300 and ˜500, respectively (Biagini, et al., 2003) the CAR ratio of T16 in yeast was estimated to less than 7. To determine the kinetic parameters of this transport, [³H]-T16 uptake was measured after 4 min incubation at 30° C. as a function of its extracellular concentration. The Lineweaver-Burk representation of this transport resulted in an apparent Kₘ value of 5.05±0.26 μM for [³H]-T16 and a maximum velocity Vₘₐₓ of 0.98±0.48 pmol/min×10⁷ cells (FIG. 8B). As a control, uptake of [methyl-³H]-choline in the wild-type strain was found to be carrier mediated with a Kₘ of 0.53±0.18 μM and a Vₘₐₓ of 40 pmol/min/10⁷ cells, as previously reported (Nikawa, et al., 1990)

To rule out the possible role of Hnm1 in the uptake of bisquaternary ammonium compounds into yeast cells, the transport of [³H]-T16 in hnm1□strain, which lacks the choline transporter gene HNM1 (FIG. 8C) was measured, and compared it to that measured in the wild-type strain (FIG. 8B). As in the wild-type strain, T16 transport in the hnm1□strain was found to be carrier-mediated with a Kₘ of 7.45±1.98 μM and a Vₘₐₓ of 0.76±0.21 pmol/min/10⁷ cells (FIG. 8C). Thus, no differences in T16 uptake could be detected between hnm1□ and wild-type strains. As expected, no choline transport could be detected in the hnm1□. Altogether, these data suggested that yeast cells utilize other transport systems for the uptake of bis-quaternary ammonium compounds.

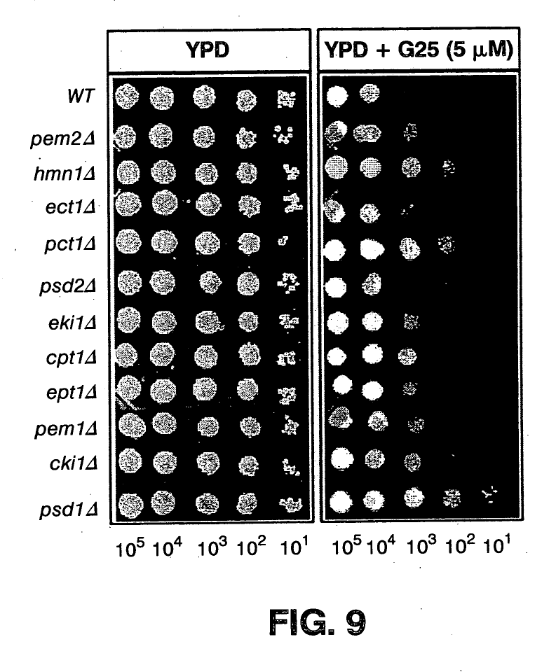

Sensitivity of mutants affected in phospholipid metabolism to choline analogs. Choline analogs have been proposed to inhibit membrane biogenesis in P. falciparum (Ancelin, et al., 2003). However, the steps in the phospholipid biosynthesis pathways that are specifically targeted by these compounds are not yet known. To assess whether the mode of action of G25 is linked to disruption of phospholipid metabolism, yeast was used as a surrogate system to compare the sensitivity to G25 of the wild-type strain and eleven individual knockouts in the CDP-choline, CDP-ethanolamine and CDP-DAG pathways. As shown in FIG. 9, substantial resistance to G25 was conferred by loss of the choline kinase (Cki1), choline phosphotransferase (Cpt1), phosphocholine cytidylyltransferase (Pct1) and choline carrier (Hnm1) activities of the CDP-choline pathway. Surprisingly, psd1□strain, which lacks the PSD1 gene encoding the PtdSer decarboxylase activity that converts 95% of cellular PtdSer into PtdEtn in the mitochondria was also found to be highly resistant to G25 (FIG. 9). No resistance was conferred by loss of the PtdEtn methyltransferases, Pem1 and Pem2, or the enzymes of the CDP-ethanolamine pathway. Furthermore, unlike psd1□ strain, loss of the Golgi/vacuole PtdSer decarboxylase, Psd2, which synthesizes 5% of the PtdEtn pool only, had no effect on G25 sensitivity. These data indicate that the sensitivity of yeast to G25 requires a functional de novo CDP-choline pathway for synthesis of PtdCho from choline and a functional Psd1 activity for PtdEtn synthesis from PtdSer.

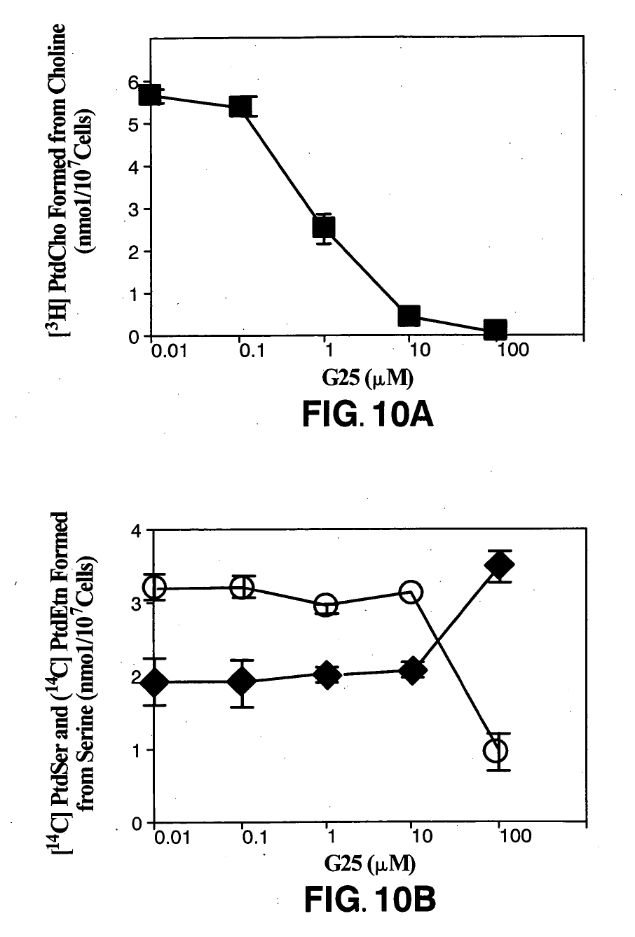

G25 inhibits the CDP-choline pathway and PtdEtn formation from PtdSer in P. falciparum. The similarity between P. falciparum and S. cerevisiae phospholipid metabolic pathways and the finding that deletion of numerous genes of phospholipid metabolism in yeast resulted in a major resistance to G25, suggested that this compound might directly inhibit phospholipid synthesizing enzymes in P. falciparum. To investigate the possible inhibition by G25 of the de novo CDP-choline pathway in P. falciparum, the incorporation of labeled choline into PtdCho in trophozoite-infected erythrocytes in the absence or presence of increasing concentrations of G25 was examined. This assay takes into account both the inhibitory effect of G25 on choline uptake as well as any additional inhibition by this compound of one or multiple enzymes of the CDP-choline pathway. As shown in FIG. 10A, G25 induced a dose-dependent inhibition of the de novo synthesis of PtdCho. At concentrations higher than 0.1 μM, G25 caused a significant decrease of PtdCho biosynthesis with 56% inhibition at 1 μM and nearly complete inhibition at 10 μM. In contrast, under similar conditions, G25 concentrations up 100 μM had no effect on the incorporation of radiolabeled ethanolamine into PtdEtn. These results are consistent with data in yeast, which showed that deletion of EK1, ECT1 and EPT1, involved in the de novo synthesis of PtdEtn from ethanolamine did not confer resistance to G25 (FIG. 9).

The possible inhibition of PtdSer decarboxylase activity in P. falciparum by G25 was investigated. P. falciparum-infected erythrocytes were labeled with radiolabeled serine, which is readily incorporated into PtdSer, in the presence or absence of increasing concentrations of G25, and the effect of this compound on the parasite endogenous PtdSer decarboxylase activity was measured by following the formation of PtdEtn from PtdSer (FIG. 10B). PtdEtn was constantly formed from PtdSer in the absence or presence of low concentrations of G25. Although the total incorpration of serine was not affected by G25, high concentrations of this compound resulted in a dramatic decrease in the endogenous PtdSer decarboxylase activity with, 77% decrease in the pool of PtdEtn formed at 100 μM G25 (FIG. 10B). Concomitantly, at this concentration of G25, PtdSer was increased in the same range indicating that the PtdSer decarboxylase activity was blocked (FIG. 10B).

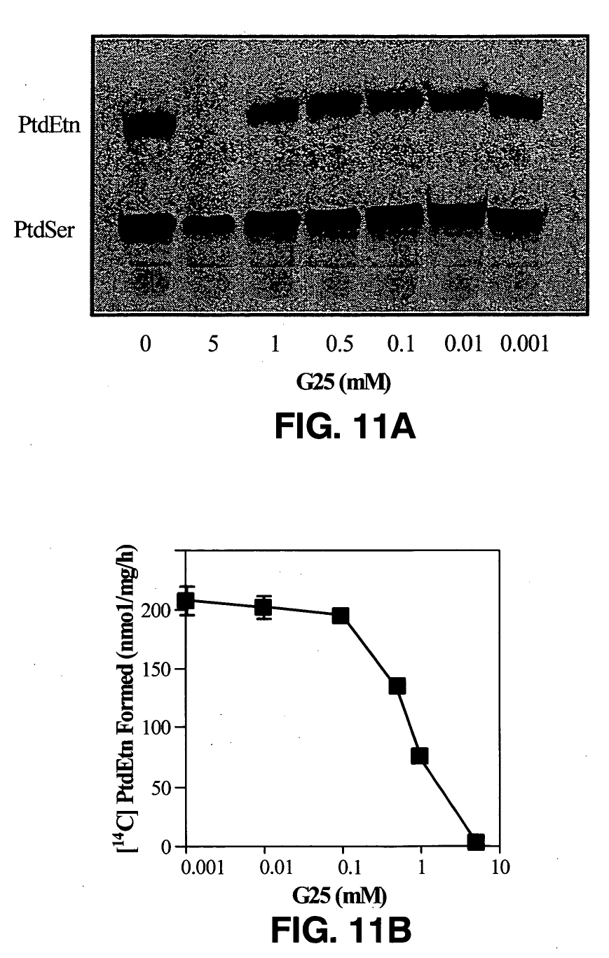

G25 inhibits the activity of recombinant P. falciparum PtdSer decarboxylase. To further investigate the inhibitory effect of G25 on the formation of PtdEtn from PtdSer, in vitro assays were performed using recombinant PtdSer decarboxylase enzyme, PfPsd1, from P. falciparum encoded by the single-copy gene, PfPSD1. The enzymatic activity of the PfPsd1 recombinant protein was tested under optimal conditions as described in Material and Methods, in the absence or presence of increasing concentrations of G25. Whereas in the absence of G25 recombinant PfPsd1 efficiently converted PtdSer into PtdEtn, addition of G25 resulted in a steady decrease in the activity of the enzyme as the concentration of the compound increased (FIG. 11A and 11B), suggesting a direct inhibition of the PfPsd1 activity by G25.

Discussion of Results

Quaternary ammonium compounds analogs of choline represent a new class of drugs with promising therapeutic future for treatment of multidrug-resistant malaria (Ancelin, et al., 1998) and possibly other parasitic infections (Zufferey, et al., 2004). Previous studies in P. falciparum have suggested that choline transport might be the primary target of these compounds; however, the role of choline influx and PtdCho biosynthesis in parasite development and survival has not been detailed. Furthermore, the difficulty to genetically manipulate P. falciparum has severely hampered efforts to understand the exact mode of action of these compounds.

For the first time, evidence is provided that the anti-malarial choline analog G25 inhibits the growth of S. cerevisiae and that mutations in phospholipid metabolic genes affect the sensitivity of yeast to this compound. The yeast and malarial metabolic pathways of phospholipid biogenesis are similar enough that the targets of phospholipid inhibitors found in yeast are most likely to be relevant to P. falciparum. The IC₅₀ value measured in yeast is 2.5 μM, whereas that measured in various P. falciparum strains ranged between 1 and 5.3 nM. Interestingly, whereas G25 and its analog T16 accumulate in P. falciparum-infected erythrocytes with cellular accumulation ratios (CAR) after 3 h incubation of ˜300 and ˜500, respectively, results indicate a CAR ratio of T16 in yeast of less than 7. The differences in growth inhibition assays and drug cellular accumulation could thus account for the differences in IC₅₀s between the two organisms.

The sensitivity of yeast to G25 and its structural analog T16, and the availability of a radioactive form of T16 prompted investigation of the effect of those two compounds on the entry of choline into S. cerevisiae. Similar to previous studies in P. falciparum, results showed that G25 and T16 are very effective inhibitors of choline transport in yeast with 50% inhibition of choline uptake measured when G25 and T16 were present in 20- and 100-fold excess, respectively. Because choline is not essential for yeast growth, and the fact that the IC₅₀ values of G25 and T16 were not affected by the presence or absence of choline in the medium, the ability of G25 to inhibit choline transport cannot alone account for its anti-fungal activity.

The entry of the G25 analog T16 in wild-type and hnm1□yeast strains has been shown to occur through a temperature-dependent carrier-mediated process with similar kinetic characteristics indicating a mode of entry of bis-quaternary ammonium in yeast distinct of the choline carrier. Daves and Krupka (1979) showed that the lengthening of the alkyl chain in choline alalogs makes them high-affinity inhibitors of choline transport, but prevents their entry via the erythrocytic choline carrier. A similar mechanism may account for the ability of G25 and T16 to inhibit choline transport in S. cerevisiae and P. falciparum without being transported via the endogenous choline carriers. In yeast, and most likely in P. falciparum as well, G25 is not transported via the choline transporter Hnm1, and once inside the cell, this compound exerts its activity by interfering with specific cellular functions.

The data showed that yeast mutants lacking specific phospholipid synthesizing genes display substantial resistance to G25. Interestingly, loss of every gene of the de novo CDP-choline pathway, choline transporter (HNM1), choline kinase (CKI1), choline phosphotransferase (CPT1) and phosphocholine cytidylyltransferase (PCT1) resulted in resistance to this compound Remarkably, a strain psd1Δ, which lacks the gene PSD1, was also found to be highly resistant to G25. In yeast, PtdSer, which is synthesized in the endoplasmic reticulum (ER) and mitochondria-associated membrane (MAM), is first transported to the inner mitochondrial membrane and Golgi/vacuole compartments, the sites of PtdSer decarboxylase 1 (Psd1p) and 2 (Psd2), respectively. It is subsequently converted to PtdEtn. Psd1p is the major PtdSer decarboxylase, converting 95% of the cellular PtdSer and producing most of the cellular PtdEtn in the absence of an ethanolamine precursor. In addition to its role in the yeast membrane structure, PtdEtn plays a central role in lysosome/vacuole autophagy by covalently conjugating to Apg8p and also serves as a donor of ethanolamine phosphate to glycosylphosphatidylinositol anchors, whose synthesis is essential for yeast cell viability. Because P. falciparum possesses homologs of the yeast PSD1, CKI1, CPT1 and PCT1 genes, the inventor hypothesized that G25 might exert its anti-malarial activity by blocking the synthesis of PtdCho from choline, and PdtEtn from PdtSer.

Labeling studies in P. falciparum, using the phospholipid precursors choline and serine demonstrated that G25 inhibited both the incorporation of choline into PtdCho and PtdSer decarboxylation in a dose-dependent manner. Only 1 μM of this compound was sufficient to inhibit PtdCho synthesis from choline, and inhibition was complete at 10 μM G25. Although this inhibition could be accounted for solely by the ability of choline analogs to inhibit choline entry into Plasmodium-infected erythrocytes, additional inhibition by this compound of one or multiple enzymes of the CDP-choline pathway may be involved. Nonetheless, G25 concentrations up to 100 μM had no effect on the de novo biosynthesis of PtdEtn from ethanolamine in P. falciparum, suggesting that the effect of this compound on the de novo PtdCho biosynthetic pathway is very specific. Similarly, albeit at higher concentrations, G25 was able to effect the incorporation of serine into PtdEtn via the CDP-DAG pathway by specifically inhibiting the decarboxylation step of PtdSer into PtdEtn. At a concentration of 100 μM, G25 inhibited PtdEtn formation from PtdSer by 77%. Interestingly, at this concentration, G25 had no effect on the first step of the CDP-DAG pathway catalyzed by the PtdSer synthase.

Two possible hypotheses could account for the resistance of yeast mutants to G25. First, G25 might not directly kill yeast, but rather be converted into toxic derivatives by Psd1 and other enzymes of the CDP-choline pathway. Deletion of the genes encoding those enzymes reduces the toxicity of the compound. Second, G25 might directly inhibit specific enzymes of the phospholipid metabolic pathways, and deletion of PSD1 or any of the four genes of the CDP-choline pathway, although not essential for survival, results in changes in the composition and/or structure of the yeast membranes leading to low entry and/or effect of G25. In yeast, PtdCho can be synthesized either via the CDP-choline pathway from choline transported via the choline transporter Hnm1, or via the transmethylation of PtdEtn by two methyltransferases encoded by PEM1/CHO2 and PEM2/0P13 genes.

The genes involved in these pathways are highly regulated by the availability of the phospholipid precursors inositol and choline Yeast cells utilize the CDP-DAG pathway as the primary route of synthesis of PtdCho. The CDP-choline pathway, although not essential, is also active even in the absence of choline in the medium. This suggests that although both pathways can compensate for each other to allow survival, the composition of PtdCho synthesized by each pathway might be different under normal conditions. Considering the mechanism of catalysis of choline kinase, phosphocholine cytidyltransferase, CDP-choline phosphotransferase and PtdSer decarboxylase, it is difficult to envisage that G25 could be a substrate for those enzymes. Furthermore, previous studies in P. falciparum using a radioactive analog of G25, VB5-T, have shown that this compound is not metabolized, and that it directly acts as an active compound (48). The in vitro studies using recombinant PfPsd1 showed that G25 specifically inhibited the PtdSer decarboxylation reaction catalyzed by this enzyme, thus providing further support for the second hypothesis.

The recent discovery in P. falciparum of a plant-like pathway for PtdCho biosynthesis involving methylation of phosphoethanolamine into phosphocholine by a phosphoethanolamine methyltransferase, PfPmt, suggests that choline uptake might not be essential for parasite survival, whereas the later steps of the CDP-choline pathway catalyzed by phosphocholine cytidyltransferase and CDP-choline phosphotransferase enzymes might be essential.

Two new mechanisms of action of G25 in P. falciparum and S. cerevisiae have been determined. G25 specifically inhibits the de novo synthesis of PtdCho from choline, and the PtdSer decarboxylase-dependent formation of PtdEtn from PtdSer. These novel findings constitute important information for quaternary ammonium compounds that are entering clinical studies. These studies further support the use of quaternary ammonium compounds previously found to be effective an antimalarials, for potential as wide spectrum antifungal compounds.

All references cited in this specification are herein incorporated by reference to the same extent as if each individual reference were specifically and individually indicated to be incorporated by reference.

### REFERENCES

U.S. Pat. No. 6,096,788 (Aug. 1, 2000)
U.S. Pat. No. 5,834,491 (Nov. 10, 1998)
WO 01/05742 (Jan. 25, 2001)
WO 2004/009068 (Jan. 29, 2004)
FR 2 842 423 (Jan. 23, 2004)
Ancelin, M. L., M. Calas, J. Bompart, G. Cordina, D. Martin, M. Ben Bari, T. Jei, P. Druilhe, and H. J. Vial. 1998. Antimalarial activity of 77 phospholipid polar head analogs: close correlation between inhibition of phospholipid metabolism and in vitro Plasmodium falciparum growth. Blood 91:1426-37.
Ancelin, M. L., and H. J. Vial. 1986. Quaternary ammonium compounds efficiently inhibit Plasmodium falciparum growth in vitro by impairment of choline transport. Antimicrob Agents Chemother 29:814-20.
Ancelin, M. L., H. J. Vial, M. Calas, L. Giral, G. Piquet, E. Rubi, A. Thomas, W. Peters, C. Slomianny, S. Herrera, and et al. 1994. Present development concerning antimalarial activity of phospholipid metabolism inhibitors with special reference to in vivo activity. Mem Inst Oswaldo Cruz 89 Suppl 2:85-90.
Ancelin, M. L., H. J. Vial, and J. R. Philippot 1985. Inhibitors of choline transport into Plasmodiuminfected erythrocytes are effective antiplasmodial compounds in vitro. Biochem Pharmacol 34:4068-71.
Ancelin, M. L., M. Calas, A. Bonhoure, S. Herbute, and H. J. Vial. 2003. In vivo antimalarial activities of mono- and bis quaternary ammonium salts interfering with Plasmodium phospholipid metabolism. Antimicrob Agents Chemother 47:2598-605.
Ancelin, M. L., M. Calas, V. Vidal-Sailhan, S. Herbute, P. Ringwald, and H. J. Vial. 2003. Potent inhibitors of Plasmodium phospholipid metabolism with a broad spectrum of in vitro antimalarial activities. Antimicrob Agents Chemother 47:2590-7.
Atkinson, K. D., B. Jensen, A. I. Kolat, E. M. Storm, S. A. Henry, and S. Fogel. 1980. Yeast mutants auxotrophic for choline or ethanolamine. J Bacteriol 141:558-64.
Baunaure, F., P. Eldin, A. M. Cathiard, and H. Vial. 2004. Characterization of a non-mitochondrial type I phosphatidylserine decarboxylase in Plasmodium falciparum. Mol Microbiol 51:33-46.
Biagini, G. A., E. Richier, P. G. Bray, M. Calas, H. Vial, and S. A. Ward. 2003. Heme binding contributes to antimalarial activity of bis-quaternary ammoniums.

Antimicrob Agents Chemother 47:2584-9.
Bimer, R., M. Burgermeister, R. Schneiter, and G. Daum. 2001. Roles of phosphatidylethanolamine and of its several biosynthetic pathways in Saccharomyces cerevisiae. Mol Biol Cell 12:997-1007.
Calas, M., M. L. Ancelin, G. Cordina, P. Portefaix, G. Piquet, V. Vidal-Sailhan, and H. Vial. 2000. Antimalarial activity of compounds interfering with Plasmodium falciparum phospholipid metabolism: comparison between mono- and bisquaternary ammonium salts. J Med Chem 43:505-16.
Calas, M., G. Cordina, J. Bompart, M. Ben Bari, T. Jei, M. L. Ancelin, and H. Vial. 1997. Antimalarial activity of molecules interfering with Plasmodium falciparum phospholipid metabolism. Structure-activity relationship analysis. J Med Chem 40:3557-66.
Carman, G. M., and S. A. Henry. 1999. Phospholipid biosynthesis in the yeast Saccharomyces cerevisiae and interrelationship with other metabolic processes. Prog Lipid Res 38:361-99.
Clancey, C. J., S. C. Chang, and W. Dowhan. 1993. Cloning of a gene (PSD1) encoding phosphatidylserine decarboxylase from Saccharomyces cerevisiae by complementation of an Escherichia coli mutant. J Biol Chem 268:24580-90.
Deves, R., and R. M. Krupka. 1979. The binding and translocation steps in transport as related to substrate structure. A study of the choline carrier of erythrocytes. Biochim Biophys Acta 557:469-85.
Englund, P. T. 1993. The structure and biosynthesis of glycosyl phosphatidylinositol protein anchors. Annu Rev Biochem 62:121-38.
Folch, J., Lees, M. and Sloane Stanley, G. H. 1957. A simple method for the isolation and purification of total lipids from animal tissues. J. Biol. Chem. 226:497 -509.
Gardner, M. J., N. Hall, E. Fung, O. White, M. Berriman, R. W. Hyman, J. M.

Carlton, A. Pain, K. E. Nelson, S. Bowman, I. T. Paulsen, K. James, J. A. Eisen, K. Rutherford, S. L. Salzberg, A. Craig, S. Kyes, M. S. Chan, V. Nene, S. J. Shallom, B. Suh, J. Peterson, S. Angiuoli, M. Pertea, J. Allen, J. Selengut, D. Haft, M. W. Mather, A. B. Vaidya, D. M. Martin, A. H. Fairlamb, M. J. Fraunholz, D. S. Roos, S. A. Ralph, G. I. McFadden, L. M. Cummings, G. M. Subramanian, C. Mungall, J. C. Venter, D. J. Carucci, S. L. Hoffman, C. Newbold, R. W. Davis, C. M. Fraser, and B. Barrell. 2002. Genome sequence of the human malaria parasite Plasmodium falciparum. Nature 419:498-511.
Hjelmstad, R. H., and R. M. Bell. 1987. Mutants of Saccharomyces cerevisiae defective in sn-1,2-diacylglycerol cholinephosphotransferase. Isolation, characterization, and cloning of the CPT1 gene. J Biol Chem 262:3909-17.
Hjelmstad, R. H., and R. M. Bell. 1991. sn-1,2-diacylglycerol choline- and ethanolaminephosphotransferases in Saccharomyces cerevisiae. Nucleotide sequence of the EPT1 gene and comparison of the CPT1 and EPT1 gene products. J Biol Chem 266:5094-103.
Hjelmstad, R. H., and R. M. Bell. 1990. The sn-1,2-diacylglycerol cholinephosphotransferase of Saccharomyces cerevisiae. Nucleotide sequence, transcriptional mapping, and gene product analysis of the CPT1 gene. J Biol Chem 265:1755-64.
Hjelmstad, R. H., and R. M. Bell. 1988. The sn-1,2-diacylglycerol ethanolaminephosphotransferase activity of Saccharomyces cerevisiae. Isolation of mutants and cloning of the EPT1 gene. J Biol Chem 263:19748-57.
Hosaka, K., T. Kodaki, and S. Yamashita. 1989. Cloning and characterization of the yeast CKI gene encoding choline kinase and its expression in Escherichia coli. J Biol Chem 264:2053-9.
Ichimura, Y., T. Kirisako, T. Takao, Y. Satomi, Y. Shimonishi, N. Ishihara, N. Mizushima, I. Tanida, E. Kominami, M. Ohsumi, T. Noda, and Y. Ohsumi. 2000. A ubiquitin-like system mediates protein lipidation. Nature 408:488-92.
Jakovcic, S., G. S. Getz, M. Rabinowitz, H. Jakob, and H. Swift. 1971. Cardiolipin content of wild type and mutant yeasts in relation to mitochondrial function and development. J Cell Biol 48:490-502.
Kim, K., K. H. Kim, M. K. Storey, D. R. Voelker, and G. M. Carman. 1999. Isolation and characterization of the Saccharomyces cerevisiae EKI1 gene encoding ethanolamine kinase. J Biol Chem 274:14857-66.
Kim, K. H., D. R. Voelker, M. T. Flocco, and G. M. Carman. 1998. Expression, purification, and characterization of choline kinase, product of the CKI gene from Saccharomyces cerevisiae. J Biol Chem 273:6844-52.
Kiyono, K., K. Miura, Y. Kushima, T. Hikiji, M. Fukushima, I. Shibuya, and A. Ohta. 1987. Primary structure and product characterization of the Saccharomyces cerevisiae CHO1 gene that encodes phosphatidylserine synthase. J Biochem (Tokyo) 102:1089-100.
Kodaki, T., and S. Yamashita. 1989. Characterization of the methyltransferases in the yeast phosphatidylethanolamine methylation pathway by selective gene disruption. Eur J Biochem 185:243-51.
Lambros, C., and J. P. Vanderberg. 1979. Synchronization of Plasmodium falciparum erythrocytic stages in culture. J Parasitol 65:418-20.
Letts, V. A., L. S. Klig, M. Bae-Lee, G. M. Carman, and S. A. Henry. 1983. Isolation of the yeast structural gene for the membrane-associated enzyme phosphatidylserine synthase. Proc Natl Acad Sci U S A 80:7279-83.
Martin, K. 1969. Effects of quaternary ammonium compounds on choline transport in red cells. Br J Pharmacol 36:458-69.
Menon, A. K., and V. L. Stevens. 1992. Phosphatidylethanolamine is the donor of the ethanolamine residue linking a glycosylphosphatidylinositol anchor to protein. J Biol Chem 267:15277-80.
Nikawa, J., K. Hosaka, Y. Tsukagoshi, and S. Yamashita. 1990. Primary structure of the yeast choline transport gene and regulation of its expression. J Biol Chem 265:15996-6003.
Nikawa, J., Y. Tsukagoshi, T. Kodaki, and S. Yamashita. 1987. Nucleotide sequence and characterization of the yeast PSS gene encoding phosphatidylserine synthase. Eur J Biochem 167:7-12.
Patton-Vogt, J. L., P. Griac, A. Sreenivas, V. Bruno, S. Dowd, M. J. Swede, and S. A. Henry. 1997. Role of the yeast phosphatidylinositol/phosphatidylcholine transfer protein (Sec14p) in phosphatidylcholine turnover and INO1 regulation. J Biol Chem 272:20873-83.
Pessi, G., G. Kociubinski, and C. B. Mamoun. 2004. A pathway for phosphatidylcholine biosynthesis in Plasmodium falciparum involving phosphoethanolamine methylation. Proc Natl Acad Sci U S A 101:6206 -6211.
Santiago, T. C., and C. B. Mamoun. 2003. Genome expression analysis in yeast reveals novel transcriptional regulation by inositol and choline and new regulatory functions for Opi1p, Ino2p, and Ino4p. J Biol Chem 278:38723-30.
Storey, M. K., K. L. Clay, T. Kutateladze, R. C. Murphy, M. Overduin, and D. R. Voelker. 2001. Phosphatidylethanolamine has an essential role in Saccharomyces cerevisiae that is independent of its ability to form hexagonal phase structures. J Biol Chem 276:48539-48.
Sullivan, D. J., Jr., I. Y. Gluzman, D. G. Russell, and D. E. Goldberg. 1996. On the molecular mechanism of chloroquine's antimalarial action. Proc Natl Acad Sci U S A 93:11865-70.
Trager, W., and J. B. Jensen. 1976. Human malaria parasites in continuous culture. Science 193:673-5.
Trotter, P. J., J. Pedretti, and D. R. Voelker. 1993. Phosphatidylserine decarboxylase from Saccharomyces cerevisiae. Isolation of mutants, cloning of the gene, and creation of a null allele. J Biol Chem 268:21416-24.
Trotter, P. J., J. Pedretti, R. Yates, and D. R. Voelker. 1995. Phosphatidylserine decarboxylase 2 of Saccharomyces cerevisiae. Cloning and mapping of the gene, heterologous expression, and creation of the null allele. J Biol Chem 270:6071-80.
Trotter, P. J., and D. R. Voelker. 1995. Identification of a non-mitochondrial phosphatidylserine decarboxylase activity (PSD2) in the yeast Saccharomyces cerevisiae. J Biol Chem 270:6062-70.
Vial, H. A. C., M. 2001. Inhibitors of phospholipid metabolism. Human Press Inc.,:347-365.
Vial, H. J., M. J. Thuet, M. L. Ancelin, J. R. Philippot, and C. Chavis. 1984. Phospholipid metabolism as a new target for malaria chemotherapy. Mechanism of action of D-2-amino-I-butanol. Biochem Pharmacol 33:2761-70.
Vial, H. 1996. Recent developments and rationale towards new strategies for malarial chemotherapy. Parasite 3:3-23.
Vial, H. J., and M. L. Ancelin. 1998. Malaria lipids, p. 159-175. In I. W. Sherman (ed.), Malaria: Parasite Biology Pathogenesis and Protection. American Society for Microbiology.
Vial, H. J., and M. Calas. 2001. Inhibitors of phospholipid metabolism, p. 347-365. In P. Rosenthal (ed.), Antimalarial Chemotherapy, mechanisms of action, modes of resistance, and new directions in drug development. Humana Press, Totowa, N.J.
Viudes, A., J. Peman, E. Canton, P. Ubeda, J. L. Lopez-Ribot, and M. Gobernado. 2002. Candidemia at a tertiary-care hospital: epidemiology, treatment, clinical outcome and risk factors for death. Eur J Clin Microbiol Infect Dis 21:767-74.
Wengelnik, K., V. Vidal, M. L. Ancelin, A. M. Cathiard, J. L. Morgat, C. H. Kocken, N I. Calas, S. Herrera, A. W. Thomas, and H. J. Vial. 2002. A class of potent antimalarials and their specific accumulation in infected erythrocytes. Science 295:1311-4.
WHO. 2000. WHO Expert Committee on Malaria. World Health Organ Tech Rep Ser 892:i-v, 1-74.
Yamashita, S., and K. Hosaka. 1997. Choline kinase from yeast. Biochim Biophys Acta 1348:63-9.
Zufferey, R., and C. B. Mamoun. 2002. Choline transport in Leishmania major promastigotes and its inhibition by choline and phosphocholine analogs. Mol Biochem Parasitol 125:127-34.
Zufferey, R., T. C. Santiago, V. Brachet, and C. Ben Mamoun. 2004. Reexamining the Role of Choline Transporter-Like (CTL) Proteins in Choline Transport. Neurochem Res 29.
Geldenhuys W J, et al., P R, Nguyen T H, Van der Schyf C J, Crooks P A, Dwoskin L P, Allen D D. 3D-QSAR study of bis-azaaromatic quaternary ammonium analogs at the blood-brain barrier choline transporter. Bioorg Med Chem. Jul. 1, 2005. 13 (13):4253-61.
Biagini G A, Pasini E M, Hughes R, De Koning H P, Vial J H, O'Neill P M, Ward S A, Bray P G, Characterizztion of the choline carrier of Plasmodium falciparum: a route for the selective delivery of novel antimalarial drugs. Blood. Nov. 15, 2004; 104(10):3372-7. Epub Jun. 17, 2004.
Calas M, Cordina G, Bompart J, Ben Bari M, Jei T, Ancelin M, Vial H., Antimalarial activity of molecules interfering with Pasmodium falciparum phospholipid metabolism. Structure-activity relationship analysis, J Med Chem. Oct. 24, 2004; 40(22):3557-66.
Salom-Roig X J, Hamze A, Calas M, Vial H J, Dual molecules as new antimalarials. Comb Chem. High Throughoutput Screen. Feb. 8, 2005. (1):49-62. Review.
Roggero R, Zufferey R, Minca M, Richier E, Calas M, Vial H, Ben Mamoun C., Unraveling the mode of action of the antimalarial cholie analog G25 in Pasmodium falciparum and Saccharomyces cerevisiae., Antimicrob Agents Chemother. Aug. 48, 2004 (8):2816-24.
Ancelin M L, Calas, M, Bompart J, Cordina G, Martin D, Ben Bari M, Jei T, Druilhe P, Vial H J. Antimalarial activity of 77 phospholipid polar head analogs: close correlation between inhibition of phospholipid metabolism and in vitro Pasmodium falciparum growth. Blood. Feb. 15, 1998; 91(4):1426-37.

What is claimed is:

1. A method for treating or preventing a fungal infection comprising administering to a host in need of therapy or prophylaxis of the fungal infection, an effective amount of a pharmaceutical composition comprising an alkyl mono- or bis-quaternary ammonium compound having the formula I:
R₁R₂R₃N⁺(CH₂)ₙR mX⁻ I
Where R is hydrogen, phenyl, alkyl, alkenyl, alkynyl, alkylimine or R₁R₂R₃N⁺—, R₂ and R₃ can combine with the quaternary nitrogen to form a heterocyclic ring selected from the group consisting of pyrrolidine, pyrrole, pyrimidine, pyridine, thiazole, thiophene, thianyl, oxolanyl, imidazole and substituted derivatives thereof wherein said substituents are selected from the group consisting of alkyl C₁-C₅ and hydroxyalkyl for C₁-C₅; n is 1-18, m is 1 or 2 and X⁻ is halide, tosylate or pharmaceutically acceptable esters, salts, solvates, clathrates or prodrugs thereof.

2. The method of claim 1 wherein the alkyl bis-quaternary ammonium compound is selected from the group consisting of compounds having the formula II,
R₁R₂R₃N⁺(CH₂)nN⁺R₄R₅R₆ mX⁻ II
wherein n is 2-18, R₁R₂R₃R₄R₅R₆ are independently alkyl, alkenyl or alkynyl; except when R₁—R₂ or R₄—R₅ are methylene; R₃ and R₆ are CH₂CH₂OH or CH₂CH₂OCH₃; and X⁻ is halide, tosylate or a pharmaceutically acceptable salt thereof.

3. The method of claim 1 wherein the alkyl bis-quaternary ammonium compound is selected from the group consisting of 1,16-hexadecylmethylenebis-[N-methylpyrrolidine], 1,12-dodecanemethylene bis[4-methyl-5-ethylthiazoline] and pharmaceutically acceptable salts thereof.

4. The method of claim 1 wherein the alkyl bis-quaternary ammonium compound is N,N,N,N-tetraethyl-N,N-di(2-hydroxyethyl)-1,16-hexadecanediaminium dibromide.

5. The method of claim 1 wherein the alkyl bis-quaternary ammonium compound is 1,16-hexadecamethylene bis-[N-methylpyrrolidinium]dibromide (DTAB).

6. The method of claim 1 wherein the alkyl mono-quaternary ammonium compound is selected from the group consisting of compounds having the formula III,
R₁R₂R₃N⁺(CH₂)n X⁻ III
wherein n is 2-18, R₁R₂R₃ are independently alkyl or alkenyl; R₁is alkyl when R₁—R₂ is methylene; R₃ is —CH₂CH₂OH or CH₂CH₂OCH₃ when R₁ and R₃ are alkyl; and X⁻ is halide, tosylate or pharmaceutically acceptable salts thereof.

7. The method of claim 1 wherein the alkyl mono-quaternary ammonium compound is 1-dodecanemethylene [N-methylpyrrolidine] or trimethyl-octadecylmethylene-amidine and salts thereof.

8. The method of claim 1 wherein the host is a mammalian host.

9. The method of claim 1 wherein the fungal infection is a Candida, Aspergillus, Coccididomycosis (Coccidioides immitis), Filobasidiella neoformans, Blastomycesdermatitidis, Paracoccidioides bresiliensis, Sporothrix schenckii, hormodendrum pedrosoi and Rhinosporidium seeberi infection.

10. The method of claim 1 wherein the fungal infection is a Candida albicans or Saccharomyces cerevisiae infection.

11. An antifungal composition comprising a mono-or bis-quaternary alkyl ammonium compound having the formula IV:
R₁R₂R₃N⁺(CH₂)nN⁺R₁R₂R₃ mX⁻ IV
Wherein R₁R₂R₃R₄R₅R₆ are independently alkyl, alkenyl or alkynyl; except when R₁—R₂ and R₄—R₅ are independently methylene, R₃ and R₆ are independently alkyl; m is 1 or 2, n is 6-18, and X⁻ is halide or tosylate.

12. The antifungal composition of claim 11 wherein the bis-quaternary alkyl ammonium compound is 1,12-dodecanemethylene bis [4-methyl-5-ethylthiazolium]diodide.

13. A pharmaceutically acceptable antifungal composition comprising at least two of the mono- or bis-quaternary alkyl ammonium compounds of claim 1.

14. The antifungal composition of claim 11 further comprising an antiflammatory compound.

15. A non-toxic systemically administerable antifungal composition comprising an alkyl mono-or bis-quaternary ammonium choline analog compound wherein the analog consists of a long chain fatty acid having from 8-16 carbon atoms and which is substituted on either end with a quaternary nitrogen.

16. A pharmaceutical antifungal tablet composition comprising a compound of formula I or a physiologically tolerable salt thereof in an amount suitable for oral administration to a mammalian host and which is comprised within a suitable timed release excipient.

17. A pharmaceutically acceptable anti-trypanosomal or anti-Leishmanial composition comprising a mono- or bis-quaternary alkyl ammonium compound of claim 1.

18. A method of treating a fungal infection comprising administering to a host in need of therapy or prophylaxis thereof, an effective amount of a pharmaceutically acceptable composition comprising any of the group of mono-quaternary ammonium compounds having the formula V:
R(CH₂)nN⁺R₁R₂R₃ X⁻ V
Where R₁ is alkyl, R₂ is alkenyl or alkyl, R₃ is branched alkyl or alkenyl or (CH₂Y)s where s is 1-12 and Y is hydroxy or hydroxyphenyl, n is 6-16, R is H or phenyl, and X⁻ is halide, OTs⁻ or pharmaceutically acceptable salt thereof.

19. A method of treating a fungal infection comprising administering to a host in need of therapy of prophylaxis thereof, an effective amount of a pharmaceutical composition comprising any of the group of bis-quaternary ammonium compounds having the formula VI:
R₁R₂R₃N⁺(CH₂)nN⁺R₁R₂R₃ mX⁻ VI
Where R₁ and R₂ are independently CH₃ and C₂H₅; R₃ is C₁-C₁₁, alkyl, alkenyl or alkynyl, m is 1 or 2, n is 6-21; and X⁻ is halide, OTs—or pharmaceutically acceptable salt thereof.

20. The method of claim 18 or claim 19 wherein the fungal infection is systemic, mucosal or topical.

21. A method for inhibiting fungal growth, comprising applying to an area, surface, material or object exhibiting presence of a fungus, a composition comprising one or more of an alkyl mono- or bis-quaternary ammonium compound of claim 1.

22. The method of claim 21 wherein the applying is accomplished by spraying or soaking the area, surface, material or object affected by the fungal presence or the fungal growth.

23. A method for treating or inhibiting a Trypanosomiasis or Leishmaniasis infection, comprising administering to a subject in need thereof a pharmaceutically acceptable composition comprising a mono- or bis-alklyammonium compound of claim 1.

24. The method of claim 23 wherein the infection is visceral or cutaneous.

25. The method of claim 23 wherein the composition comprises 1,16-hexadecamethylene bis-[N-methylpyrrolidinium] dibromide (DTAB).

26. A packaged formulation for use in treating fungal or Leishmaniasis infections comprising a pharmaceutical composition comprising a mono- or bis-alkylammonium compound of claim 1 and instructions for use.
